(19) United States
(12) Patent Application Publication
(10) Pub. No.: US 2010/0324440 A1
(43) Pub. Date: Dec. 23, 2010

(54) REAL TIME STIMULUS TRIGGERED BY BRAIN STATE TO ENHANCE PERCEPTION AND COGNITION

(75) Inventors: Christopher Irwin Moore, Cambridge, MA (US); Mark Lawrence Andermann, Brookline, MA (US)

Correspondence Address: DITTHAVONG MORI & STEINER, P.C., 918 Prince Street, Alexandria, VA, 22314, US

(73) Assignee: Massachusetts Institute of Technology, Cambridge, MA (US)

(21) Appl. No.: 12/488,416

(22) Filed: Jun. 19, 2009

(51) Int. Cl. A61B 5/0484

(52) U.S. Cl. 600/544

(57) ABSTRACT

An approach is provided for real time stimulus triggered by brain state and includes receiving data that indicates a brain state and a set of one or more stimuli associated with the brain state. Onset of an instance of the brain state is detected in a subject. In response to detecting onset of the instance, application to the subject of a stimulus of the set is initiated before the instance ends. In some embodiments, the brain state is determined based on a range of values for a function of brain signal data, wherein the range of values is associated with desired performance in response to an associated stimulus. The approach can enhance performance, enhance learning or enhance the probing of impact of that state on perception, action or cognition.

### COPYRIGHT NOTICE

This patent application contains material subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

The brain's interpretation of sensory stimuli at any given time can rely heavily on the subject's instantaneous brain activity, or ‘brain state.’ Such states are observed at multiple temporal and spatial scales. Much progress has been made in understanding the perceptual effects of variability in sensory brain responses measured in a time interval after presentation of a target stimulus, called “stimulus-locked” sensory brain response. Brain states prior to stimulus onset have also been studied, and in many cases are correlated with the success of cognitive performance (e.g., successful use of memory), perception (e.g., perceiving accurately the stimulus presented during a given state) and of motor action (e.g., success at initiating movement).

However, systemic study of rare patterns of ongoing activity remains elusive because they rarely coincide with target stimulus presentation, and are not under the control of the experimenter. Any advantage for a subject to enhance response as a result of a desirable pre-target brain state is thus difficult to exploit.

### SOME EXAMPLE EMBODIMENTS

Therefore, there is a need for ways to exploit rare but desirable states of brain activity for enhancing response. The enhanced response includes enhanced detection, enhanced learning or enhanced performance, alone or in some combination.

According to a first set of embodiments, a method includes receiving data that indicates a brain state and a set of one or more stimuli associated with the brain state. Onset of an instance of the brain state is detected in a subject. In response to detecting onset of the instance, application to the subject of a stimulus of the set is initiated before the instance ends.

In some of these embodiments, detecting the onset of the instance of the brain state includes determining that a value of a function of one or more electrical signals detected at corresponding electrodes placed near the subject falls within a predetermined range of values.

In some embodiments of the first set, the brain state is associated with a superior capacity by the subject to perceive a particular sensory input. In some embodiments of the first set, the brain state is associated with a superior capacity by the subject to perform a particular function.

In some embodiments of the first set, the brain state is more likely to occur in response to a particular stimulus, and the method further comprises initiating application to the subject of the different stimulus.

In a second set of embodiments, a method includes receiving signal data and performance data. The signal data indicates one or more electrical signals detected at corresponding electrodes placed near a first subject. The performance data indicates response of the first subject to a stimulus during a time interval encompassed by the signal data. Desired performance within the performance data is determined. A brain state is determined based on a range of values for a function of the signal data, wherein the range of values is associated with the desired performance. The stimulus is presented to a second subject when the brain state is detected in the second subject.

According to other sets of embodiments, a computer-readable storage medium, or apparatus is configured to perform one or more steps of the above embodiments.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings, in which:

FIG. 1A is a diagram that illustrates a system for deriving brain states, according to one embodiment;

FIG. 1B is a diagram that illustrates a module for detecting brain signals, according to one embodiment;

FIG. 1C is a graph that illustrates changes in brain states, according to an embodiment;

FIG. 2A is a diagram that illustrates a system for triggering a stimulus based on brain state, according to an embodiment;

FIG. 2B is a diagram that illustrates a system for triggering an audio stimulus based on brain state, according to an embodiment;

FIG. 3 is a flowchart that illustrates a process for deriving brain states associated with enhanced performance, according to one embodiment;

FIG. 4 is a flowchart that illustrates a process for triggering a stimulus based on brain state for enhanced performance, according to one embodiment;

FIG. 5 is a diagram that illustrates alternative timings for performance training, including an embodiment;

FIG. 6 is a graph that illustrates advantage of performance training based on brain state-triggered stimulus, according to various embodiments;

FIG. 7 is a graph of stimuli presented to a subject during derivation of brain states and performance training, according to various embodiments;

FIG. 8 is a diagram that illustrates performance detection, according to an embodiment;

FIG. 9A is a graph that illustrates an index for determining brain state associated with performance, according to an embodiment;

FIG. 9B is a graph that illustrates index evolution with time in a subject, according to an embodiment;

FIG. 9C is a graph that illustrates average index evolution with time in a subject, according to an embodiment;

FIG. 9D is a graph that illustrates extreme variation among three different trials of index evolution with time in a subject, according to an embodiment;

FIG. 10 is a graph that illustrates index thresholds to define two brain states, according to an embodiment;

FIG. 11A is a graph that illustrates cue-induced incidence of brain states among multiple subjects, according to an embodiment;

FIG. 11B is a graph that illustrates distribution among subjects of excess incidence of brain states consistent with cue, according to an embodiment;

FIG. 11C is a graph that illustrates correct performance as a function of brain state and cuing, according to an embodiment;

FIG. 11D is a graph that illustrates a performance error as a function of brain state and cuing, according to an embodiment;

FIG. 12 is a graph that illustrates elevated high gamma activity increases miss rate regardless of cueing, according to an embodiment;

FIG. 13 is a diagram of hardware that can be used to implement an embodiment of the invention; and

FIG. 14 is a diagram of a chip set that can be used to implement an embodiment of the invention.

### DETAILED DESCRIPTION OF EXAMPLE EMBODIMENTS

A method, apparatus, and software are disclosed for real time stimulus during brain state associated with stimulus. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

In conventional usage within the neuroscience community, ‘brain state’ is often taken to mean the sustained maintenance of an oscillatory brain rhythm, such as those named by Greek letters alpha, beta, gamma, etc. Here, we use the term ‘brain state’ in a more general sense to indicate any pattern of brain activity that indicates the timing is optimal for stimulus presentation to accelerate performance, learning or experimental design. Examples that transcend he narrow conventional definition of brain state include patterns that are indicated by the convergence of classical ongoing rhythmic activity patterns (e.g., gamma in one brain area with alpha in another), or a pattern of progression of oscillatory patterns (e.g., if gamma just occurred and has now ceased, the post-gamma period may be optimal, but only at a certain latency to the preceding expression of oscillatory activity). As indicated elsewhere, such marker states may also be detected using non-electrical means, such as patterns of blood flow and volume

Although several embodiments of the invention are discussed with respect to a brain state associated with unilateral aural attention and real-time unilateral auditory stimulus to improve perception of the stimulus (as expressed in detection rates by the subject), embodiments of the invention are not limited to this context. It is explicitly anticipated that in other embodiments, the brain state is associated with the same or different capacity to attend to, perceive, perform, learn, remember phenomena external to the subject, or perform some cognitive or other bodily function internal to the subject, such as moving a particular muscle; and the stimulus is, is correlated with, is contrary to, or is an alert for the external phenomena or function associated with the brain state. Various embodiments serve different training purposes, from remembering information presented or forgetting stored information, to controlling amplified or dampened perception of sensory input, to amplifying or dampening the external phenomena for a sensory aid or prosthesis, to controlling increased or decreased movement or other bodily function. Thus, in various embodiments, an association between change in brain state and change in ability of a subject is used to direct training of the same or similar subject, or to direct operation of sensory aids or prostheses for the same or similar subject, or both.

Given the importance of ongoing brain states for cognition, perception and action, timing the presentation of stimuli to different specific states could have 3 primary benefits, described next, among others.

A first benefit results from timing stimulus presentation to a specific brain state to enhance performance. As one example, timing the presentation of auditory output of a cell phone to the listener's preparedness to hear a given input could enhance listening capability.

A second benefit results from timing stimulus presentation to a specific brain state to enhance learning. As one example, timing the presentation of a phoneme in a foreign language to a brain state when the listener is likely to hear the distinction may accelerate her ability to learn that distinction. As one example, teaching a Japanese listener the distinction between the English ‘L’ and ‘R’ sounds could be made possible or the learning rate may be accelerated by timing these stimuli during learning to the relevant state. As a second example, in stroke rehabilitation from motor deficit, timing the instruction to move to the time period when the subject has a brain state in which the he is likely to be successful in moving may make possible or accelerate his ability to move.

A third benefit results from timing stimulus presentation to a specific brain state to enhance the probing of impact of that state on perception, action or cognition. The systemic study of rare patterns of ongoing activity remains elusive because they rarely coincide with target stimulus presentation, and are not under the control of the experimenter. As one example, if a hearing aid were trying to learn the brain states that corresponded to the need for louder stimulus presentation, the training of the automated detection of a brain state that required a different amplitude of presentation by the device could be made possible or accelerated by this mechanism. As a second example, neuroscientific research studies of the meaning of specific and more rare brain states could be made possible or accelerated by this approach.

Example mal-adaptive brain conditions that may benefit from brain-state triggered stimuli to enhance training include dyslexia, attention-deficit/hyperactivity disorder (AD/HD or ADHD), autism, brain injury and stroke damage, among others. Example mal-adaptive neural conditions that may benefit from brain-state triggered stimuli to enhance training of motor skills include Parkinson's and nerve injury. Example mal-adaptive sensory systems that may benefit from brain-state triggered stimuli to enhance training or operation of sensory assist devices, such as hearing aids, include hearing loss and vision impairment, due a variety of disease, injury or age, among others, alone or in any combination. Other benefits would accrue to subjects with normal brain conditions and sensory systems, such as normal subjects attempting to learn a new skill or language, especially one with elements not already in the student subject's repertoire, such as a distinction between the sounds of the English letters “l” and “r” for a person raised only exposed to the Japanese language.

In other applications, the brain state triggered stimulus can be used as a method to present critical stimuli at just the right time in a environment dense with information (called “external stimulus flooding”), e.g., for fighter pilot, air traffic controller, stock broker, among other professions. In other applications, the brain state triggered stimulus can be used in situations where intense concentration on one task can occlude attention to external stimuli and therefore the cause the stimuli to be lost, e.g. for race car driver, special operations agent, air traffic controller, astronaut, among other professions. In other applications the brain state triggered stimulus can be used in training animals, e.g. for movies and entertainment shows and other circumstances where training with an improved success rate can translate into considerable time and revenue savings, thus justifying the efforts involved. For example, in movies several animals have to be trained to do the same tricks (for backup purpose) for one and the same shot.

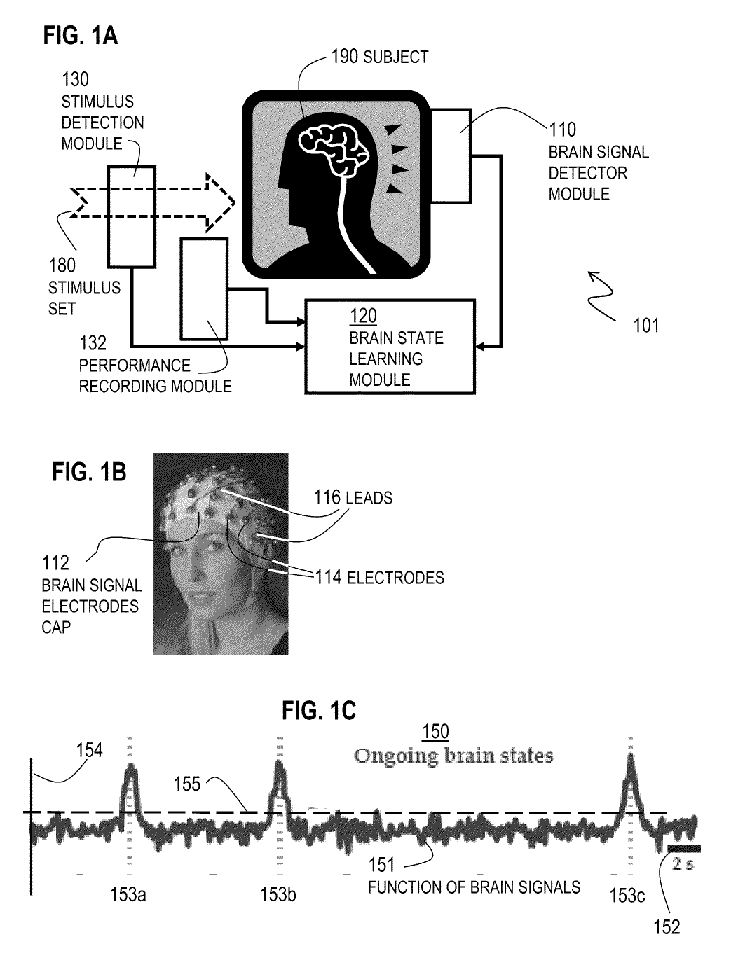

FIG. 1A is a diagram that illustrates a system for deriving brain states, according to one embodiment. These brain states are associated with some enhanced capability of the subject to sense, learn or act as determined from performance observations. The system includes a brain signal detector module 110, a brain state learning module 120, a stimulus detection module 130 and a performance recording module 132. The system 101 operates on a subject 190 who is exposed to a stimulus set 180 of one or more stimuli, including any optional alerts or cuing to induce a favorable brain state. In response to the stimulus set 180, the subject performs some task, such as indicating perception of sensory input by lifting a finger or attempting to perform some function, such as taking a walking stride. Some or all of the stimulus set 180 is detected by the stimulus detection module 130; and the performance is recorded by the performance recording module 132 while the brain signal detector module 110 is detecting brain signals from subject 190. The brain state learning module 120 is configured to associate values of one or more functions of the brain signals with the performance recorded, and to determine a range of values for one or more functions that is correlated with good performance or anti-correlated with bad performance, or both. The range of values for the one or more functions constitutes a brain state associated with performance. Thus, as used herein, a brain state is a one-sided or two-sided range of values for one or more functions of one or more measured brain signals. In some embodiments, measurement of correlated physiological signals, such as a change in skin conduction or pupil diameter, are also employed in determining an optimal state for stimulus presentation.

In various embodiments, human input by one or more researchers is involved in one or more of stimulus detection module 130, performance recording module 132 and brain state learning module 120. In some embodiments, the brain signal detection module 110, stimulus detection module 130, performance recording module 132 and brain state learning module 120 are all configured to be fully automatic—not requiring human input or other human intervention.

In various embodiments, the brain signal detector module 110 is any device that detects activity in the brain, including electrical, magnetic, thermal and chemical activity using sensors near the subject's brain, including sensors at, on or below the subject's scalp and indirect measurements of brain activity, e.g., measurements of pupil dilation or skin conductance.

FIG. 1B is a diagram that illustrates a module for detecting brain signals, according to one embodiment. In the illustrated embodiment, the brain signal detector 110 includes the brain signal electrodes cap 112, with electrodes 114 to detect temporal changes in electrical potential at corresponding locations on the subject's scalp. Typically, a highly conductive connection between electrode and scalp is made as follows: First, hair is moved out of the way, and any dry scalp is gently scraped. Next, a small amount of electrode paste is applied between the scalp and the electrode contact. Typically, electrode impedances lower that 5-10 thousand Ohms (kiloOhms, kOhms) are desired. For example, the ACTICAP™ from BRAIN PRODUCTS™ of Gilching, Germany is depicted in FIG. 1B and provides electro-encephalography (EEG) electrical signals on 32 channels corresponding to 32 electrodes distributed over the cap 112. Wire leads 116 transmit the measured potential to a signal conditioning and recording device, not shown, that is part of the brain signal detector module 110. Many such signal conditioning and recording devices are known in the art, for example an electro-encephalograph or BRAINVISION™ Recorder, from Brain Products.

In other embodiments other sensors and sensor arrangements are used as brain signal detector module 110, such as a multi-electrode array of invasive implanted electrodes, or a magneto-encephalography (MEG) device, well known in the art. An example of multielectrode devices is the NEUROPORT™ Array available from CYBERKINETICS NEUROTECHNOLOGY SYSTEMS, INC.™ of Foxborough, Mass., USA. An example of MEG devices is Elekta NEUROMAG™, from ELEKTA™ AB, Norcross, Ga., USA.

The stimulus detection module 130 is configured to detect some or all stimuli of the stimulus set 180 presented to the subject 190. In some embodiments, the stimulus detection module 130 is configured to generate some or all of the stimulus set 180; and detection involves simply recording the generation of the corresponding stimulus. In some embodiments, a human operator inputs data indicating the time or type or both of a stimulus set.

The performance recording module 132 is configured to record the performance of the subject in detecting the sensory input or performing the action in response to the stimulus set. In some embodiments, a human operator observes the response of the subject and enters data indicating the response into the module 132. In some embodiments, the performance recording module 132 is also configured to detect the performance, such as the desired response by the subject, e.g., by detecting the subject depressing a key or gripping a pressure sensitive handle or lifting an instrumented load, in response to the stimulus set 180. In various embodiments, video or audio equipment is used to capture the response; and, in some of these embodiments, recognition logic is employed to determine automatically whether the video or audio recording displays the desired performance.

The brain state learning module 120 is configured to determine functions of the brain signal supplied by the brain signal detector module 110 for which values are correlated with desired performance. Any function may be used. In some embodiments, signals from one or more sensors are correlated individually with the desired performance and one or more of the most highly correlated signals are weighted and summed to produce a weighted sum that correlates highly with the desired performance. In some embodiments, arbitrary functional forms (e.g., polynomial, trigonometric, and transcendental functions) or principal components are fit to the performance data to obtain a best match, as is well known in the art of curve fitting. One or more values for the functions of the brain signal input, expressed as open-ended (one-sided) or closed (two-sided) ranges, are associated with the desired performance. For embodiments in which multivariate functions are used, a range of values can be expressed as a cluster in multidimensional space, as is well known in the art. In some embodiments, human intervention is involved in determining the ranges associated with desired performance. In some embodiments, some or all of the steps for determining the ranges associated with desired performance are performed automatically without human intervention.

In some embodiments, the desired performance is not observed directly by the performance recording module 132 but is inferred from other observations. For example, in an illustrated embodiment described in more detail in the next section, the desired performance is an enhanced ability to detect a deviant tone in a series of tones, but the observed performance is an indication of enhanced attention to the one ear where the deviant tone will be presented. It is assumed that enhanced attention to the correct ear is associated with the desired performance to enhance detection of the deviant tone.

In some embodiments, after a brain state associated with desirable performance is determined, different stimuli are presented to the subject to determine if the frequency of occurrence of the brain state is affected by the different stimuli. If so, the different inducing stimulus is also learned in the brain state learning module 120 and is included in a subsequent stimulus set 180. For example, as described in more detail in the next section with reference to the illustrated embodiment, a series of staggered tones in both ears along with a visual cue to attend to one ear is more likely to generate enhanced attention to the correct ear and the associated brain state.

FIG. 1C is a graph 150 that illustrates changes in brain states, according to an embodiment. The horizontal scale is indicated by bar 152 that corresponds to a duration of two seconds. The vertical axis 154 indicates amplitude of a function of one or more brain signals. For purposes of illustration, it is assumed that the trace 151 is a weighted sum of EEG voltage from one or more electrodes in cap 112 that was correlated with performance. It is further assumed for purposes of illustration that function values above a threshold amplitude 155, indicated by a dashed line, are well correlated with desired performance. Thus brain state instances 153a, 153b, 153c, and others, not shown, collectively referenced hereinafter as brain states 153, occur at times when the function value is above the threshold 155 and are times when the subject 190 was better able to perform as desired, e.g., detect sensory input or perform some action, e.g., take a stride. In some embodiments, the brain state onset is marked by a first threshold and brain state end is marked by a different second threshold, e.g., a lower threshold.

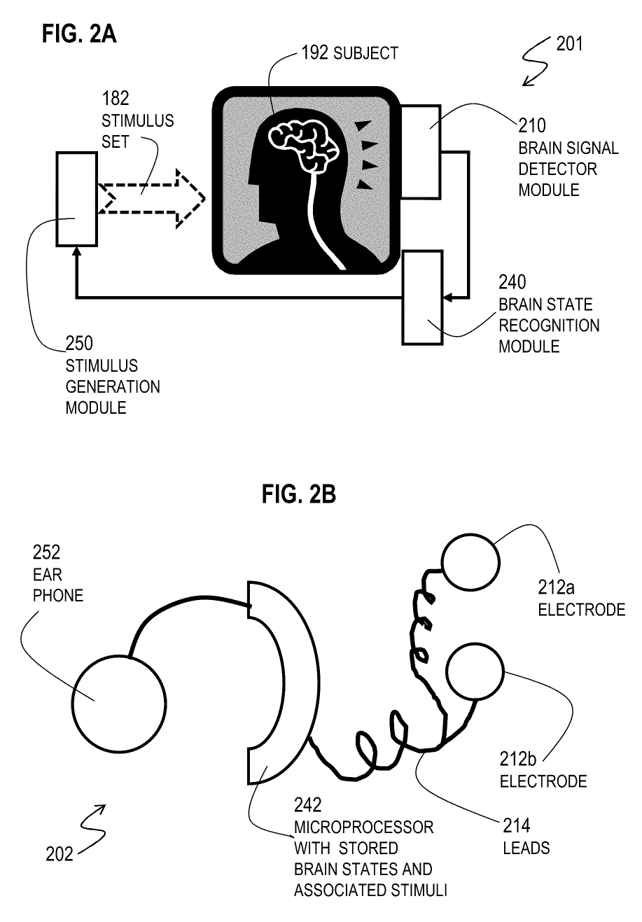

FIG. 2A is a diagram that illustrates a system 201 for triggering a stimulus based on brain state, according to an embodiment. The system 201 includes a brain signal detector module 210, a brain state recognition module 240, and a stimulus generation module 250. The system 201 operates on a subject 192 who is exposed to at least one stimulus in some stimulus set 182 in response to a brain state recognized in module 240 based on data received from brain signal detector module 210. In response to the stimulus set 182, the subject performs some task, such as indicating perception of sensory input by lifting a finger or attempting to perform some function, such as taking a walking stride. Since the brain state is associated with an enhanced capability to perform the task, the effectiveness of the stimulus set 182 is increased. In some embodiments the stimulus set includes a different inducing stimulus to induce the brain state associated with enhanced capability, such as the visual cue and series of staggered tones in the illustrated embodiment described in the next section. In such embodiments, only the deviant tone is included in the stimulus set in response to the brain state; the other stimuli in the stimulus set 182 are presented on some other schedule.

In some embodiments, the brain states are very personal to a subject, and the subject 192 is the same as subject 190 for whom the brains states were derived. In some embodiments, the brain states are more generally applicable to multiple subjects in the same general or specific population category, and the subject 192 may be different from the subject 190.

In some embodiments, the system 201 includes a performance detection module 132, as depicted in FIG. 1A, to determine the performance. The detected performance is used in some embodiments to derive an improved brain state, or determine a modified stimulus set 182. An advantage of such embodiments is to allow the brain state definition or stimulus set to evolve with changing conditions of the subject 192, due fatigue or other distraction, or due to a physiological difference from the original subject 19, when the subjects 190 and 192 are different persons.

In some embodiments, the brain signal detector module 210 is the same as brain signal detector module 110, such as a full cap 112. In some embodiments, the module 210 is different, e.g., including only the subset of electrodes that was used in the function that defines the brain state of interest and excluding other electrodes.

The brain state recognition module 240 is configured to determine the onset of a brain state, e.g., the increase of amplitude of trace 151 above threshold 155, before the instance of the brain state ends.

The stimulus generation module 250 is configured to generate the stimulus 182 upon detection by the brain state recognition module of the onset of the particular brain state. It is an advantage if the stimulus 182 is presented before the brain state ends, e.g., before the trace 151 next drops below the threshold 155, because the subject's brain is in a state, e.g., state 153, for increased capability to respond to the stimulus. In some embodiments, the stimulus generator 250, or a different stimulus generator, not shown, is configured to present a different inducing stimulus to increase the likelihood that the brain state will occur. Detection and stimulus generation on the time scale of the duration of a brain state is called real-time triggering herein

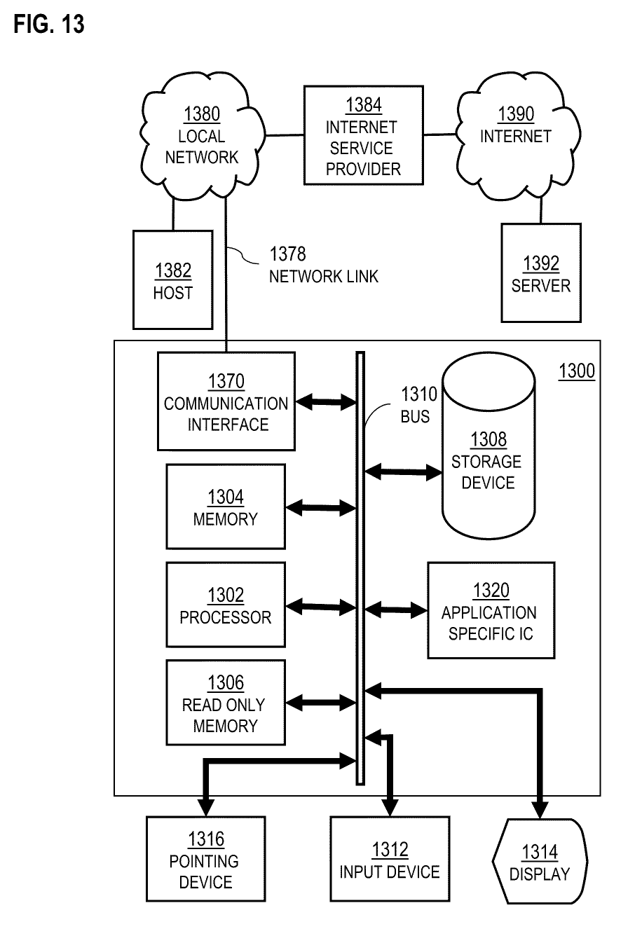

Although a particular set of separate modules are shown in FIG. 1A and FIG. 2A in a particular arrangement for purposes of illustration, in various other embodiments more or fewer modules, or portions thereof, may be included, separated, combined or arranged in some other fashion. For example, in some embodiments, one or more modules are processes executing on one or more general purpose computers or nodes on a network, as described in more detail below with reference to FIG. 13.

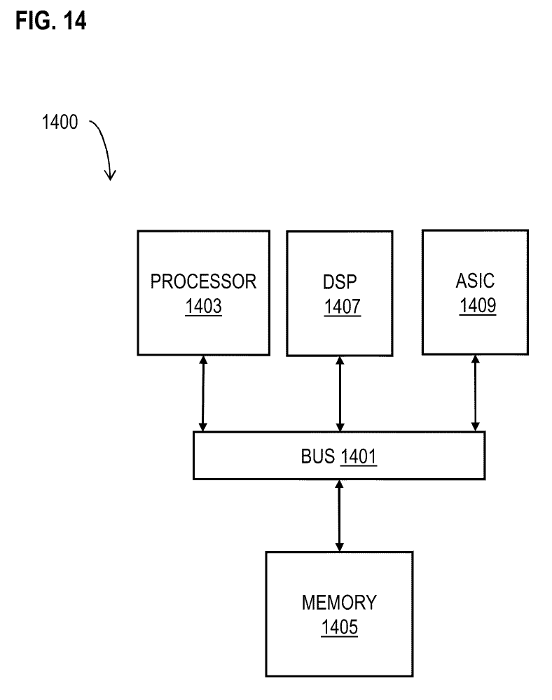

FIG. 2B is a diagram that illustrates a system 202 for triggering an audio stimulus based on brain state, according to an embodiment. The system includes electrode 212a and electrode 212b collectively referenced hereinafter as electrodes 212. The electrodes 212 are connected by leads 214 to microprocessor device 242 with stored data indicating brain states and associated stimuli. The microprocessor device 242 includes one or more chip sets as described in more detail below with reference to FIG. 14. The microprocessor device 242 drives earphone 252. Thus electrodes 212 and leads 214 constitute a particular embodiment of brain signal detector 210. Similarly, microprocessor device 242 is a particular embodiment of brain state recognition module 240; and, earphone 252 is a particular embodiment of stimulus generation module 250.

Brain state-guided stimulus presentation augments the utility of currently available sensory prostheses. For example, speech perception is often impaired in individuals that use hearing aids, particularly in noisy environments requiring increased attention. This is likely due in part to the inability of hearing aids to mimic the dynamic modulation of gain control in the inner ear. Attention related brain states, associated with feedback modulation of outer hair cells, might be specific to a given ear and even to a given sound frequency band. In addition, peripheral auditory impairment may cause a subsequent degradation in the capacity for central selective attention. A hearing aid embodiment based on system 202 provides frequency band gain (stimulus) triggered on attention-related brain states that track the biases in cortical attention to speech streams at a given ear. In this way, EEG-triggered dynamic modulation of incoming sound intensity and other sound features is used in an attention brain state guided hearing aid. This potentially helps restore the central control of peripheral auditory processing that is otherwise diminished in hearing-impaired individuals.

It is also known that presentation of a prolonged subliminal low frequency tone can be used to stimulate extension of the audible frequency band for an individual to lower frequencies. In some embodiments, instead of a prolonged emission, system 202 is used to trigger presentation of the subliminal low frequency in response to detecting a aural attention brain state. This intermittent presentation saves substantial power and may prove nearly as effective as the prolonged presentation of the known approach.

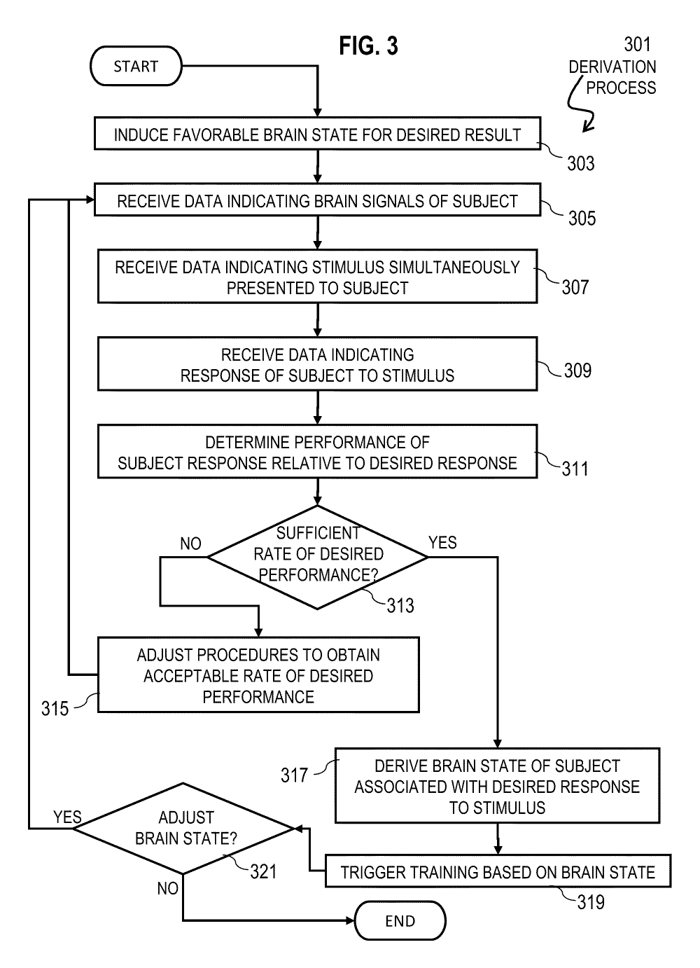

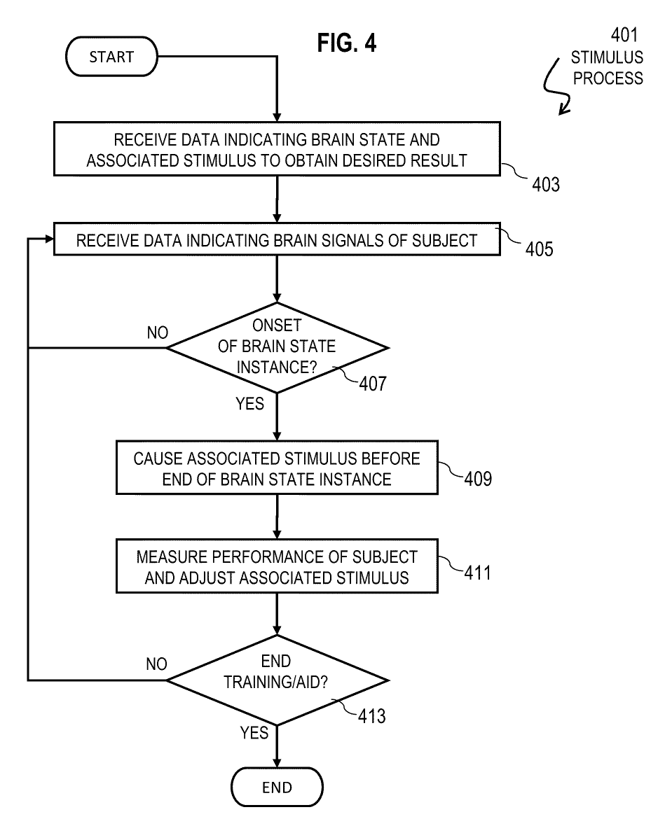

FIG. 3 is a flowchart that illustrates a process 301 for deriving brain states associated with enhanced performance, according to one embodiment;. Although steps in FIG. 3 and in subsequent flow chart FIG. 4 are shown in a particular order for purposes of illustration, in other embodiments, one or more steps may be performed in a different order or overlapping in time, in series or in parallel, or one or more steps may be omitted or added, or changed in some combination of ways. For purposes of illustration, it is assumed that brain states are to be learned for increasing a subject's ability to distinguish the English letter “L” sound from the English letter “R” sound.

In step 303, a favorable brain state for a desired result is induced. For example, the subject is cued to pay attention to some sense or body part. In an illustrated embodiment, the subject is provided with a visual cue and a series of staggered tones, using a different pitch in each ear, to increase the chances of the subject experiencing a unilateral attention brain state. In some embodiments, it is not known or possible to induce the favorable brain state and step 303 is omitted. In many cases (e.g. rehabilitation therapy), step 303 reduces to just standard attending to a sensory detection task.

In step 305, data is received indicating brain signals detected for a subject. For example, data is received indicating signals detected at one or more electrodes 114 of cap 112. Any method may be used to receive this data. For example, in various embodiments, the data is included as a default value in software instructions, is received as manual input from a system administrator on the local or a remote node, is retrieved from a local file or database, or is sent from a different node or module on a network, either in response to a query or unsolicited, or the data is received using some combination of these methods. For example, data indicating 32 different electrodes in a cap 112 are included as default values in software, while a stream of analog or digital values of electrical amplitudes at those electrodes is received from module 110.

In step 307, data is received, which indicates a particular stimulus is presented to the subject. For example data is received from stimulus detection module 130. Any method may be used to receive this data, as described above. For example the subject is presented with a visual letter “L” and the corresponding English sound followed by the visual letter “R” and the corresponding English sound, as received in default data in the software, and the timing of the presentations are received at module 120 from module 180. In another example, the subject is presented with a stimulus comprising a subliminal low frequency (e.g., at 25 Hertz, Hz, 1 Hz=1 cycle per second) to encourage sensory performance to detect sound at the lowest audible frequency range (e.g., about 40 Hz). In the illustrated embodiment, described in more detail in the next section, data is received indicating the visual cue and the start of the series of tones staggered between each ear.

In step 309, data is received, which indicates the response of the subject to the stimulus. For example, the subject is instructed to press a numeric key on a computer keyboard to indicate how different the two sounds appear, a zero indicating no difference and a 9 indicating a clear and certain difference, and intervening numbers indicating intermediate differences. As another example, the subject is instructed to press a “Y” key on a computer keyboard to indicate hearing a low frequency sound, e.g., 38 Hz, added on top of the 20 Hz signal. In some embodiments, the subject indicates the response by lifting a finger, e.g., an index finger, and an observer/operator enters the response at a keypad or computer keyboard.

In step 311, the performance of the subject is determined relative to a target response. For example, it is assumed for purposes of illustration that a target response is a response of 4 or more for the difference between L and R sounds, and a particular subject indicates a response of 4 or more only 5 percent of the time. In the other example, a target response is a simple pressing of the Y button.

In step 313 it is determined whether the rate of desired performance is sufficient. If not, then in step 315, procedures are adjusted to obtain an acceptable rate of performance. For example, if it is assumed for purposes of illustration that a higher than 5% rate of obtaining a response of 4 is desired, procedures are adjusted to try to increase the success rate, such as reversing the order of the L sound and the R sound, or preceding the sound with the video cue rather than presenting simultaneously, or increasing the amplitude or pitch of the two sounds. Control then passes back to step 305 and following to obtain better performance. In some embodiments, a sufficient rate of desired performance is not set; and steps 313 and 315 are omitted.

In some embodiments, the brain signals are correlated directly with the input stimulus rather than with observed performance; and step 309 and 311 are also omitted. For example, in the illustrated embodiment described in more detail in the next section, prior published work is used to indicate that the maximum response to a cued ear, labeled the N100 response, occurs about 100 milliseconds (ms, 1 ms=10⁻³ seconds) after a tone is presented at the cued ear. Thus, in this embodiment, the actual brain signals at about 100 ms after a tone is used as a surrogate for actual observations of a target response; and steps 309 through 315 are omitted.

In step 317, a pre-stimulus brain state, defined as a range of values of a function of one or more measured brain signals, is associated with a desired response following the stimulus, e.g. a response of 4 or more or a response of “Y” or a maximum difference in N100 signals between right and left tones, whether by positive correlation or negative correlation, immediately or after a delay. For example, after completion of an initial block of stimuli, it is determined which brain states beginning prior to presentation of stimulus actually led to desired performance. This can be achieved simply by averaging pre-stimulus activity in successful trials and separately in non-successful trials, or by more complicated inference (e.g. using principal components analysis). In some embodiments, a set of one or more stimuli are associated with the brain state to produce one or more desired responses.

In some embodiments, such as embodiments that skip steps 309 through 315, step 317 determines an indirect measure of performance, e.g., a brain state associated with a measure of attention, rather than direct performance measure. In these embodiments, brain state is chosen based on neural measure of attention, not performance. It is known from previous studies that attention (e.g. to one ear, or to the ear rather than the eye or arm) improves performance (e.g. regarding sound detection at that ear). Based on this assumption, the technique is tailored to individual subjects by learning what the attention brain state (e.g., the N100 response) averaged across trials in a block is for a given subject.

In some embodiments, step 317 is performed by deducing associations between brain state and performance based on published data; and steps 303 through 315 are omitted.

In step 319, the brain state is used to trigger the stimulus to increase the subject's chances of performing well. For example the presentation of the visual and audio representations of the English letters L and R are triggered by a brain state associated with enhanced capacity to discern a difference between them (e.g., brain states associated with response of 4 or more). A process to perform step 319 is depicted in more detail with reference to FIG. 4. It is recognized that different brains might have different patterns for the same meaning, e.g., difference between L and R, and that brain states are personal to an individual subject. It is also recognized that different brains might have similar patterns for the same meaning, e.g., attention, and that brain states derived for one subject may be used to train a different subject.

It is further recognized that optimal brain states (e.g., brain states strongly associated with superior attention or performance) are not likely to be perfectly stationary with time, and may evolve over time scales of minutes or hours or days. Thus, in some embodiments, the process includes step 321 to re-assess the pre-stimulus brain states periodically and update what the optimal state is before or during each session of brain state triggered training in step 319.

FIG. 4 is a flowchart that illustrates a process 401 for triggering a stimulus based on brain state for enhanced performance, according to one embodiment. For example, module 240 is configured to perform process 401.

In step 403, data is received, which indicates brain state and associated stimulus to obtain a desired result. For example data is received that indicates a brain state (e.g., a function identifier for a function of brain signals, and range of values) that is associated with a response of 4 or better for hearing a difference between the English letters L and R. Any method may be used to receive this data, as described above. For example, in various embodiments, the data is included as a default value in software instructions, is received as manual input from a project administrator on the local or a remote node, is retrieved from a local file or database, or is sent from a different node or module on a network, either in response to a query or unsolicited, or the data is received using some combination of these methods.

In step 405, data is received indicating brain signals detected for a subject. For example, data is received indicating signals detected at one or more electrodes 114 of cap 112. Any method may be used to receive this data, as described above. For example, data indicating a stream of analog or digital values of electrical amplitudes at select nodes in a cap 112, which are used in the function indicated in step 403, are received from module 210. In some embodiments, step 405 includes inducing the optimal brain state by presenting in the stimulus set 182 a different stimulus that increase the likelihood that the optimal brain state will occur. For example, in the illustrated embodiment described in more detail in the next section, the visual cue and series of staggered tones are presented to the subject 192.

In step 407, it is determined whether an instance of the brain state has started, e.g., whether the onset of the brain state is detected. For example, it is determined whether the weighted sum indicated by trace 151 has risen above the threshold 155. If not, then control passes back to step 405 to continue to receive data indicating the brain signals (and issuing stimulus set to induce the desired brain state, if any).

If it is determined in step 407, that the onset of the brain state is detected, then, in step 409, at least one stimulus of a set of one or more stimuli associated with the brain state is presented to the subject in real time. For example, the brain state recognition module sends data indicating the stimulus to the stimulus generation module 250 to cause the stimulus generation module to present the stimulus 182 to subject 192. For example, during brain state instance 153b, the module 240 causes the module 250 to present the visual letter L with its corresponding sound followed by the letter R with its corresponding sound to subject 192 before the end of brain state instance 153b.

It is desirable for the stimulus to be presented to the subject in real time. As used herein, real time refers to a time within the time scale of the brain state duration after onset of the brain state. In some embodiments, the presentation is made at a particular phase during the instance of the brain state. For example, if it is assumed for purposes of illustration that the brain state instance 153b is indicted by an electrical oscillation at 40 Hz above a threshold amplitude 155 for a duration of 50 cycles (e.g. for 1.25 seconds). In this embodiment, the presentation is made at a certain phase of the 40 Hz oscillation, e.g., during the upswing from low potential to high potential on at least one cycle of the 50 cycles before the end of the 1.25 seconds.

In some embodiments, the process ends after step 409. In an illustrated embodiment, the process includes step 411, in which the performance of the subject is measured. For example, it is determined whether the subject indicates a response of 4 or more in the discerned difference between the L sound and the R sound. In some embodiments, the definition of the brains state or stimulus is adjusted during step 411. For example, the amplitude or pitch of the sounds are changed, or control passes back to step 321 of FIG. 3.

In step 413, it is determined whether the training or assist to the subject is to end. If not, control passes back to step 405 to receive more data indicating brain signals of the subject.

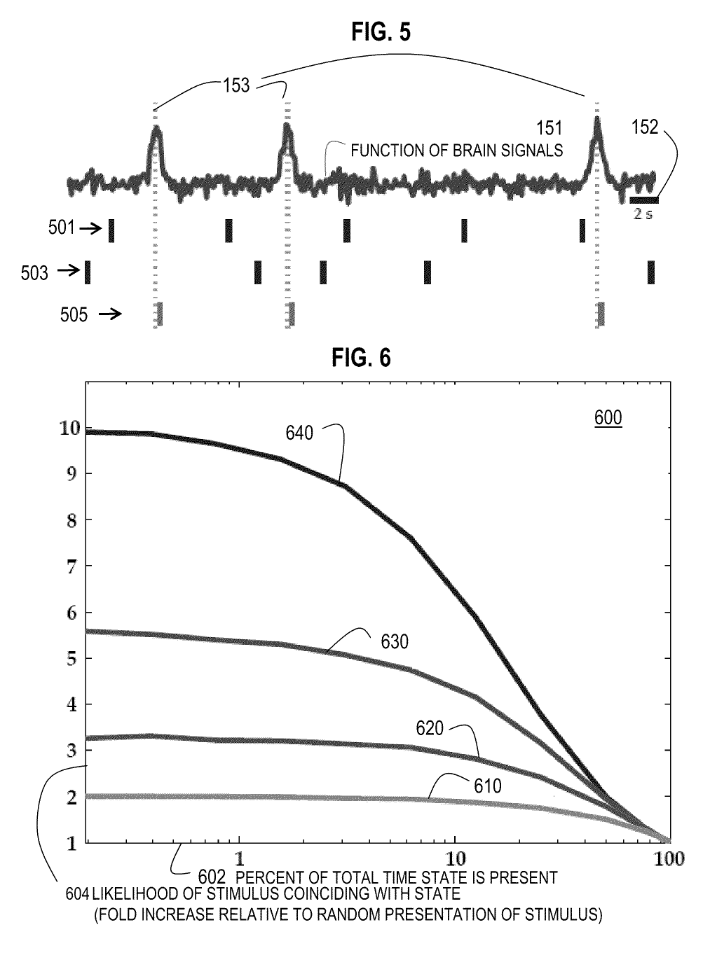

The advantages of brain state triggered stimulus presentation are made clearer with reference to FIG. 5 and FIG. 6. FIG. 5 is a diagram that illustrates alternative timings for performance training, including an embodiment. The trace 151 and time interval 152 and brain states 153 are as described above for FIG. 1C. It is assumed that performance is better when a stimulus is presented during an instance of a brain state 153.

If the stimulus (e.g., the L and R visual and audio representations) is presented at evenly spaced times with constant time intervals indicated by the vertical bars aligned with arrow 501, or at random times indicated by the vertical bars aligned with arrow 503, then there is only a small chance that the subject will be in the optimal brain state 153 associated with superior performance when the stimulus is received; and the subject's success rate will be relatively low. However, if the stimulus is presented during the optimal brain states 153, as indicated by the vertical bars aligned with arrow 505, then the subject's success rate will be relatively high. The increase in efficacy of the stimulus will not only train the subject faster by making better use of the subject's time, but by avoiding frustration or negative reinforcement that is likely to occur by the ineffective stimuli presented at times without optimal brain states, the subject is likely to be trained in many fewer repetitions of the stimulus.

The quantitative advantage of brain state triggered presentation of stimulus depends on how frequently the optimal brain state occurs and how long the optimal brain state lasts. The longer the duration and the more frequent the occurrence, the more likely a random or evenly spaced stimulus will coincide with the optimal brain state, and the lower the advantage of the brain state triggered presentation of the stimulus. However, even a small percentage increase in efficacy can be valuable. For example, a 50% increase in efficacy means that training that normally takes 3 months can be performed in two months. Saving one month of training can save thousands of dollars per trainee.

FIG. 6 is a graph that illustrates advantage of performance training based on brain state-triggered stimulus, according to various embodiments. The logarithmic horizontal axis 602 indicates the percent of total time that a brain state is present, which is equal to the average frequency of occurrence times the average duration of each occurrence. However, it is noted that the occurrence or duration or both might vary randomly and not adhere to the average frequency or duration. The vertical axis 604 indicates the increased likelihood that the brain state triggered stimulus coincides with the optimal brain state compared to a random stimulus.

Curve 610 shows the improvement achieved for a brain state with an average duration of 1000 ms. Such brain states are easily detected by properly placed, electro-encephalography (EEG) electrodes. Curves 620 and 630 show the improvement achieved for a brain state with an average duration of 800 ms and 400 ms, respectively. Such brain states are easily detected by magneto-encephalography (MEG). Curve 640 shows the improvement achieved for a brain state with an average duration of 200 ms. Such brain states are detectable using large-scale, invasive multi-electrode recordings.

Thus, brain states with about 1 second (s) duration that each occur about 25% of the time lead to a two-fold increase in likelihood of coinciding with optimal brain state, a major advance considering the limited duration of human psychophysiology experiments. Using other recording techniques that have increased signal-to-noise ratios and information rates, such as MEG and large-scale, invasive multi-electrode recordings, it is possible to utilize brain states that are more rare (present 1-5% of the time), and of more brief duration (e.g. 200 ms). For states of this nature, state-triggered stimulus presentation should afford a fivefold to tenfold increase in efficiency, with potentially transformative implications for training. For example, potential improvements in measurement and analysis techniques could allow sufficient detection of attention state using single left/right tone pairs, enabling assessment of the influence of rapid transient attention changes at the 200 ms timescale. Indeed, cued attention shifts can cause rapid changes in attention modulation of neural activity (on about a 200 ms timescale) in humans.

### DETAILED EXAMPLE EMBODIMENT

As a first proof-of-principle, this general method was applied to the use of ongoing brain dynamics in humans during a selective listening task based on EEG data. Successful implementation of brain state triggered stimulus presentation utilizes high-quality estimates of instantaneous brain states of interest within single trials. As described below, the difficult spatial detection task employed in this embodiment generates robust, selective biasing of average evoked responses to sounds presented at an attended vs. non-attended ear. The task is thus useful for studying the perceptual effects of neural bias brain states within and across single trials. The largest auditory attention modulation (and largest signal-to-noise ratio) is obtained in paradigms involving difficult target stimuli and fast sound repetition rates.

One such auditory EEG paradigm involves presentation of two rapid and independent streams of standard tones with randomized inter-tone-intervals (mean interval about 200 ms) and of differing pitch (audio frequency) at the left and right ear. In a previous study, subjects were cued to attend to a particular ear and detect rare ‘deviant’ target sounds of slightly different intensity. That study demonstrated an attention-related doubling in the average ‘N100’ EEG response (about 80 to 150 ms latency after onset of stimulus, likely localized to auditory cortex) to identical tones when attention was directed towards vs. away from the target ear. However, studies of that kind could not assess whether brain states associated with attention drifted spontaneously towards and away from the cued ear across time within single trials due to the use of randomized inter-tone intervals. More generally, such studies typically lack the statistical power to carefully examine the effects of target presentation during instances of largest neural bias towards processing of inputs from a given ear, because such instances are rare and unpredictable.

In an illustrated detailed embodiment, the above paradigm was modified to obtain a running estimate of dynamic fluctuations in ear-specific bias (called unilateral attention herein) in evoked brain signals, by presenting alternating sounds to the left and right ears using a constant inter-tone-interval. The temporal lag between stimuli allowed the separation in time of the contributions to ongoing brain signals in the N100 response from each pair of tones presented at the left and right ear. This embodiment obtains a running estimate of brain signals indicating bias towards processing sounds from a given ear. It was then determined whether fluctuations in neural bias within and across identically cued trials influenced behavioral response performance. As described below, a robust method was devised for real-time triggering of target stimuli (called deviant stimuli herein) of slightly differing intensity following the onset of an instance of a unilateral attention brain state associated with strong bias towards or away from the cued ear.

It was found that, for identical cue conditions, triggering target stimulus presentation following a strong transient brain state of correctly directed bias did influence behavioral performance, resulting in an increase in detection rates for the target stimuli, as well as an increase in false-alarm rates.

This approach of real time stimulus triggering has general applicability for efficient study of ongoing brain activity in neurons and circuits, as well as applicability for clinical applications such as the design of a hearing aid guided by an attention brain state, described above.

More specifically, in the illustrated embodiment, a GO/NOGO auditory deviant detection task experiment modified from previous studies was performed with concurrent EEG recordings, in twenty-one volunteers. Subjects were cued to attend to the left or right ear. Two spectrally separable trains of auditory tones were presented to the left and right ear at 5 tones per second (5 Hz) for five seconds. The tones were staggered by 100 ms so that ear-specific brain signals could be identified. In ˜80% of trials, one of the standard tones at the cued ear was replaced by a deviant target tone of identical frequency but slightly higher intensity. To avoid confounds in interpreting brain signals due to motor preparation, subjects were cued to wait until the stimulus train ended (5 s), and raise their right index finger to report detection of the deviant tone, followed by brief visual feedback. The possible outcomes were hit (correct detection), miss, false alarm (finger lift when no deviant tone present), and correct reject (no finger lift when no deviant present

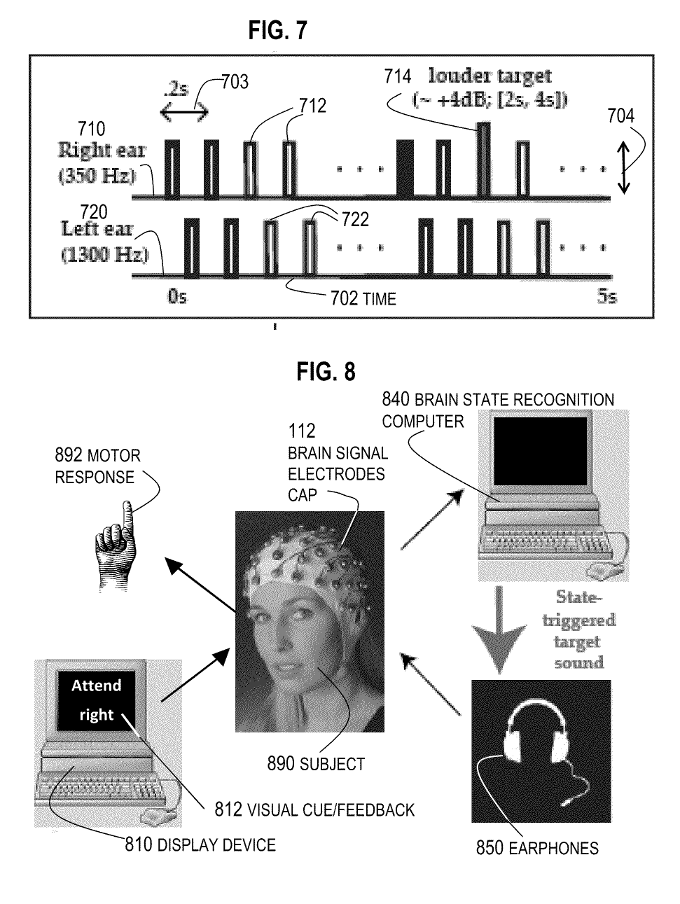

FIG. 7 is a graph of stimuli presented to a subject during derivation of brain states and performance training, according to various embodiments. The horizontal axis 702 indicates elapsed time after start of a trial, with time scale 703 corresponding to the 0.2 s (200 ms) between starts of successive tones in a single ear. The right ear was subjected to a series of 350 Hz tones 710 of approximately equal amplitude; while the left ear was subjected to a series of 1300 Hz tones 720, staggered by 0.1 s (100 ms) to occur between the 350 Hz tones. An example deviant tone 714 for the right ear is the same frequency as other tones for the same ear, but has a greater amplitude, increased by 4 decibels (dB, 1 dB=one tenth of the logarithm of a ratio between the acoustic pressure of the tone and a reference pressure)in which the reference pressure of previous standard tones. In these experiments, the visual cue and the series of tones are the stimulus associated with the unilateral attention brain state to induce the state, and the deviant tone is a second stimulus associated with the unilateral brain state to be triggered by the brain state. The subject's ability to detect the deviant tone correctly is the desired (target) response.

FIG. 8 is a diagram that illustrates performance detection, according to an embodiment. Subject 890 is equipped with a brain signal electrodes cap 112, as described above, and earphones 850, and presented with a visual cue 812 on a display device 810, such as a computer with a display screen. Brain signals are recorded on brain state recognition computer 840 and used to derive unilateral attention brain states associated with attend right and attend left stimuli, and to recognize derived brain states for triggering the target deviant tone. The attend right and attend left stimuli take the form of visual cues on display device 810 and the train of staggered tones. The attend right and attend left stimuli are surrogates for improved right detection performance and improved left detection performance, respectively. The computer 840 also drives earphones 850 worn by subject 890 to provide the stimuli.

Actual performance is determined based on detecting a motor response 892 of subject 890 in the form of a raised index finger, when the subject 890 detects a deviant tone in cued ear (The subject is told not to respond to a deviant tone in the non-cued ear). After the brain states associated with attend left and attend right are derived and stored on computer 840, the computer issues the right (or left) ear deviant tone to the earphones in real time based on detecting the attend right (or left) brain state in the signals from cap 112. The performance of subject 890 is then detected to determine the efficacy of the brain-triggered stimulus.

A single session of simultaneous psychophysics and EEG recordings was conducted for each of 21 healthy adult volunteers (17 males) following prior informed consent. All procedures were in accordance with ethics committee guidelines at the Helsinki University of Technology.

Sounds were presented in a sound-attenuated room using high-quality headphones (HD590 from Sennheiser of Old Lyme, Conn.) rated up to 48000 Hz. As depicted in FIG. 7, two perceptually distinct 5 Hz trains of ‘standard’ tone pips were presented to the left and right ear for 5 s (left ear tones at 1350 Hz, right ear tones at 350 Hz). Presentation software by Neurobehavioral Systems of Albany, Calif. was designed to allow concerted focus on a given ear. Left and right ear tone trains were staggered by 100 ms to maximally separate in time the evoked brain signals driven by left and right ear stimuli. Tones were 12 ms long, including 5 ms half-sinusoid tapers on either end.

One major difference between previous ‘dichotic’ listening tasks and the task employed in this study is the use here of fixed 5 Hz trains of standard tones to each ear, shifted between ears by 100 ms. The constant timing of left/right ear tone pairs was advantageous to obtain an ongoing estimate of selective attention that was unbiased by variable inter-tone intervals known to affect response magnitude. This modification also enabled the assessment of the dynamics of attention tuning throughout the train of tones.

A maximum brain signal response to attention at one ear was observed at about 100 ms after each tone of the series of tones and labeled the N100 response. The N100 responses (e.g. at 120 ms latency) to stimulation of one ear may also contain smaller response components due to the stimulus presented 100 ms earlier at the other ear. However, because of the larger amplitude and attention modulation of the observed N100 responses, the majority of the attention modulated signal likely arose from N100 latent brain signal activity.

As a preliminary matter, the auditory intensity of standard tones was determined for each subject by first determining ear- and tone-specific hearing thresholds using a staircase procedure. Subsequently, tone intensity in either ear was set at 60 dB above hearing threshold. Due to differential perception of high and low-frequency tones at these intensities, tone intensity was further adjusted (<4 dB) until subjects reported equal perceived intensity in either ear, thus minimizing potential systematic bias to a given ear.

In step 303 for deriving brain states and step 405 for detecting brain states, described above, subjects were presented with a visual cue (large white arrow, persisting for the duration of the trial) indicating the ear to which the subject should attend. After 400 ms, separate 5 Hz trains of standard tones were presented for 5 s to the left and right ear, staggered by 100 ms so that ear-specific response components could be identified, as depicted in FIG. 7.

In a subset of trials (about 80%), during step 405, one of the standard tones between 2 s and 4 s after the start of the train of tones was replaced by a deviant target tone of identical frequency but slightly higher intensity (e.g., deviant tone 714). After the series of tones ended, during step 309, subjects had 1200 ms to raise their right index finger to report having detection of the deviant tone. The delay of 1-3 s between target tone and motor response was important to reduce the influence of motor preparation on pre-stimulus activity. The possible trial outcomes were hit (correct detection), miss, false alarm (FA, wherein a finger lift is observed when no deviant was present) and correct reject (CR, wherein it is observed that a finger is not lifted when no deviant was present). Subjects then received visual feedback (cue arrow turns red for miss/false alarm, green for hit/correct reject), for a 200 ms duration during both training and state-triggered deviants. In general, the brain state remained stable over the course of the experiment, which indicates this brain state's utility as a robust indicator.

A typical experiment lasted 1.5 hours and consisted of 1-2 training runs, to derive the brain states according to process 301, followed by 6-8 test runs. Each run lasted seven minutes and consisted of 48 target trials (24 trials cued to each ear), and 4-15 no-target trials (called ‘catch’ trials herein). The sequence of trials consisted of alternating blocks of six trials cued to the same ear, to facilitate sustained focused attention in a given direction, and decrease spurious attention shifts related to novel cue information. Breaks between runs (2-5 minutes) enhanced sustained concentration throughout the experiment.

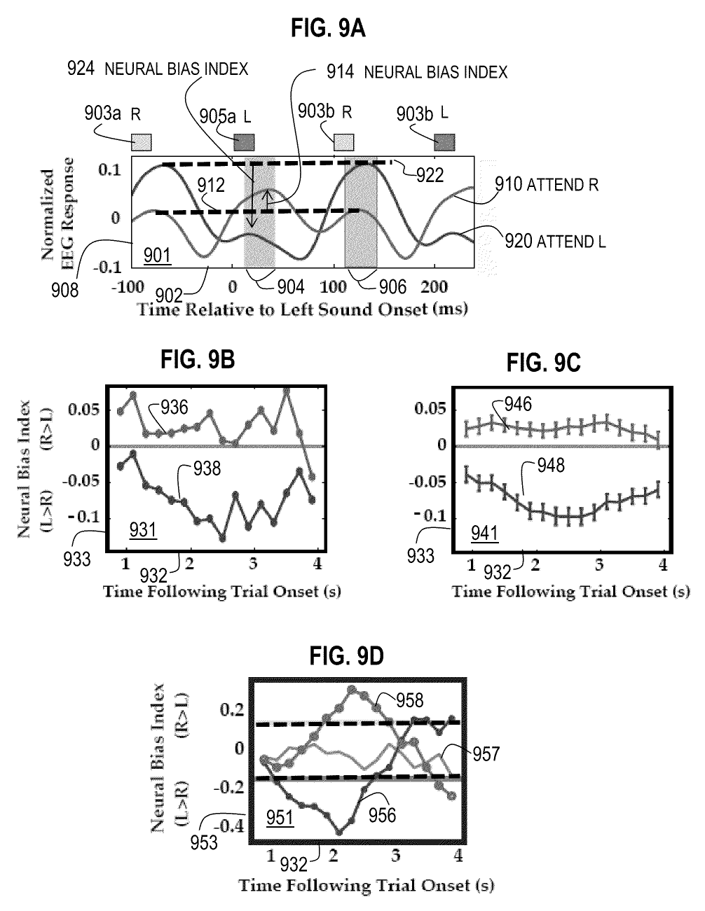

Following training runs, in step 317, average brain responses to left-ear standard tones were calculated during the attend-left and attend-right cue conditions (weighted average time series across channels, filtered and further averaged across 20 tone pips presented 1-5 s post-train-onset). For initial assessment of cue-specific task modulation of brain signal activity, these within-trial peri-tone time series were further averaged across all artifact-free trials for each cue condition. As shown for one subject in FIG. 9, described below, larger brain signal values were observed following tones at the cued ear for all 21 subjects. This suggests that cue-dependent changes in the brain signal activity reflects, in part, attention modulation of neural responses, consistent with previous studies of attention that employed similar tasks.

During steps 311 and 411 as described above, performance of the subject is determined. The demanding task employed here contained large numbers of both ‘hit’ and ‘miss’ responses. To simulate difficult tasks, such as post stroke rehabilitation or tasks far outside a user's experience, the intensity of deviant tones was adjusted separately for left and right ear tones between trials to maintain about 50% success rate (success rate=(hits+correct rejects)/(number of trials)). At the start of the first training run, during step 311, a clearly audible ( louder by >8 dB) deviant target tone replaced a standard tone at a random time during the target period (2-4 s after start of the train). For each subsequent trial, if three hits and/or CR occurred in a row, task difficulty was increased by reducing deviant intensity by 1 dB in step 315 (0.25 dB during step 413 in triggered runs). Likewise, three misses and/or FAs in a row resulted in an increase of deviant intensity by 1 dB in step 315 (0.25 dB during step 413 in triggered runs). This procedure prevented long stretches of only hits or misses, which were not included in assessments of the influence of local fluctuations in brain state on local differences in performance by the subject. Training runs were therefore extremely advantageous for subjects to reach a fairly stationary performance ‘plateau’, at which point only small intensity adjustments were made due to residual effects of learning/fatigue.

During step 305, data indicating brain signals are obtained. In the experiment, a low-noise, 32 channel EEG brain-computer interface system previously used for online brain imagery-guided cursor control in healthy subjects and tetra-pelagic patients was modified. The EEG cap (ACTICAP™) was positioned on the subject's head with a 20 cm separation between the vertex and the nasion (intersection of the frontal and two nasal bones of the human skull); and, all electrode contacts (for corresponding channels) were filled with conductive paste. Placement of the cap was accelerated by the presence of multi-colored LED lights for each electrode providing rapid feedback to indicate whether the impedance was below the 5 kiloOhm (kOhm, 1 kOhm=10³ Ohms) threshold desired. The resulting setup times were less than 15 minutes. EEG acquisition involved ‘active shielding’ for automatic reduction of estimated line noise and other external artifacts, followed by digitization at 500 Hz (BrainAmp amplifier and BrainVision Recorder software from BrainProducts of Gilching, Germany). These technological advances greatly facilitated the use of EEG in the illustrated embodiment with brain-triggered sensory feedback.

After study of brain signals associated with unilateral attention and derivation of brain states, real time estimation of brain states is employed in step 407 by module 240. In the illustrated embodiments, the Brain Products Recorder software passed EEG data from the last 2 s to MATLAB™ (available from TheMathworks of Natick, Mass.) once every 20 ms via a C/C++ computer language control program and a network connection utilizing the Transmission Control Protocol encapsulated in the Internet Protocol (TCP/IP). A MATLAB™ software program then determined whether a brain state of interest had recently occurred, prompting the C/C++ program to send a limited time to live (TTL) message as a trigger back to a computer serving as the stimulus generation module 250, and causing the next standard tone to be replaced by a deviant intensity tone with identical timing as the target stimulus to induce desired performance. The main C/C++ control program for the sensory brain-computer interface (i.e., brain recognition module 240) consisted of three threads, one for program execution, one for data acquisition from the Vision Recorder through TCP/IP, and one for signal processing and classification in MATLAB™ through a MATLAB™ Engine connection.

During derivation of the brain states, and during step 411 to adjust stimulus, the brain state learning module 120 received triggers for each tone presented (Presentation software), along with the most recent 2 s block of amplified EEG data, which was then filtered with a 4th order Butterworth filter between (2-20 Hz). To decrease artifacts, the subjects were instructed to relax their facial muscles and blink between trials. We identified eye blink, saccade and muscle artifacts as epochs where the maximum minus the minimum EEG value (in a time interval from −1.5 s to 0 s, where 0 s is the start time of the series of tones) between electrodes above and below the subject's left eye exceeded a threshold. The threshold was calculated as two standard deviations above the mean in 2-4 s intervals after start of the series of tones in the training run. Rare epochs containing artifacts were excluded from further analysis.

During step 317, in the illustrated embodiment, two brains states associated with left ear attention and right ear attention, respectively, were derived. The timing of these brain states was determined based on the visual cue given to the subject and the 200 ms of brain signals following each tone presented to the cued ear.

A measure of the overall bias in ongoing attention to left vs. right ear stimuli, termed the neural bias index (NBI), is used as the function for the brain state derived for each side, as described below. First, during the training run(s), the peak amplitude of the evoked signals (between 110-190 ms after each tone) for all 29 electrodes, averaged across 20 left and right ear tones (in the time interval from 1 to 5 s after start of the series of tones) within each trial and across all 48 trials in the training run(s). A 29×1 ‘N100’ response vector was generated from the mean of each channel in the time interval from −10 ms to 10 ms surrounding the peak N100 response. This spatial vector served as a spatial set of weights that was subsequently convolved with the incoming single-trial data to generate a single time-series on each trial. It is noted that the spatial distribution of the N100 EEG responses was qualitatively similar following left and right ear tones (data not shown), and so to simplify the computation of the NBI, left- and right-ear spatial response profiles were averaged together. Various other embodiments potentially derive more information on selective attention by using different weights for convolution with left- and right-ear responses.

One additional step used to focus the analysis to relevant EEG channels was to exclude channels that did not, on average, demonstrate clear sensitivity to attention differences during the training runs. Specifically, the measure Rbias was defined as (left-ear response—right-ear response) for each channel and single trial. The only channels used were those for which a sensitivity measure was greater than 0.15. The sensitivity measure is equal to (mean(Rbias when cue is attend left)−mean(Rbias when cue is attend right))/std(Rbias). Excluding such channels resulted in 83±13% of channels being used (mean±std. dev., N=21 subjects; 100% for subject whose data is shown in the following figures). The final weighting vector for an individual subject was then used for all test runs for that subject without modification. The resulting single time series were then averaged separately for left- and right-ear cued trials. A 30 ms time interval (centered in the time interval from 100 ms to 150 ms after the start of the train of tones) was found that generated the largest contrast between attention to left ear and attention to right ear (i.e., largest value of (Rbias when cue is attend left)−(Rbias when cue is attend right)).

The neural bias index (NBI) at any given instant is defined as the left ear response (averaged over this optimal 30 ms interval following left-ear tones) subtracted from the right ear response (averaged over the same interval following right ear tones). In some embodiments, this difference was further averaged across approximately 6 pairs of tones in the time interval from −1.5 s to −0.25 s of the current tone to increase signal-to-noise ratio. In this embodiment, the NBI is the function of brain signals for which a particular range of values defines a brain state that will trigger a deviant tone.

The fluctuations in NBI were next assessed within and across trials by calculating the NBI for each successive pair of left- and right-ear tones. FIG. 9A is a graph 901 that illustrates the index for determining brain state associated with performance, according to an embodiment. The horizontal axis 902 is time from start of a tone presented to the left ear; and the vertical axis indicates the normalized EEG response (weighted average of the used EEG channels). The timing of the left ear tones are indicated by the bars 905a and 905b above the graph 901; and the timing of the right ear tones are indicated by the bars 903a and 903b. The attend R trace 910 represents the average across the last twenty tones in each trial (in the time interval from 1 to 5 s after start of the series of tone) and across all trials in the training set where the subject is told to attend to the right ear. Similarly, attend L trace 920 represents the average across the last twenty tones in each trial (in the time interval from 1 s to 5 s after start of the series of tone) and across all trials in the training set where the subject is told to attend to the left ear. The data in graph 901 is for the one subject who showed attention sensitive signals in all 29 EEG channels, including the rare deviant tones.

The N100 response is the maximum response after start of the tone on the ear for which the subject is cued, which is at time interval 904 for attend R trace 910 and at time interval 906 for attend L trace 920, about 130 ms after the start of each tone. Note that the average response to a left-ear sound (in interval 906) is much larger when attention is cued to the left ear. Similarly, brain signal activity following right ear sounds (interval 904), were greater for the attend right condition.

The NBI can be shown on the average traces 910 and 920 for purposes of illustration, but is actually computed, when shown in following figures, on the instantaneous time series of weighted EEG signals or averaged over several previous tones. The neural bias index (NBI) was defined as the left ear response (averaged over interval 906) subtracted from the right ear response (averaged over interval 904). The left ear response in interval 906 is approximately indicated by the dashed horizontal lines 912 and 922 for the attend R trace 910 and attend L trace 920, respectively. Thus for the attend R trace 910, the NBI 914 is given by subtracting the height of line 912 from attend R trace 910 in interval 904, a positive value. Similarly, for the attend L trace 920, the NBI 924 is given by subtracting the height of line 922 from attend L trace 920 in interval 904, a negative value of much greater magnitude than for attend right (NBI 914). The NBI should be positive when instantaneous attention is directed to the right, negative for attention to the left, and near zero for split attention or low levels of attention.

FIG. 9B is a graph 931 that illustrates index evolution with time in a subject, according to an embodiment. The horizontal axis 932 indicates time following start of the series of tones, in seconds (s). The vertical axis 933 indicates the Neural Bias Index (NBI) value. Trace 936 shows the NBI for individual pairs of tones when the subject has been cued to attend right, hence the predominance of small positive values. Trace 938 shows the NBI for individual pairs of tones when the subject has been cued to attend left, hence the predominance of larger negative values. FIG. 9C is a grap 941 that illustrates average index evolution with time in a subject, according to an embodiment. Horizontal axis 932 and vertical axis are the same as in FIG. 9B. Trace 946 shows the NBI for a boxcar average of about 6 tones in a 1.25 s window preceding the plotted time when the subject has been cued to attend right. Trace 948 shows the NBI for similar averaging when the subject has been cued to attend left. Traces 946 and 948 are less noisy than their un-averaged counterparts, traces 936 and 938, respectively.

While there was some similarity in NBI evolution with time among different trials for the same subject, there were some dramatic differences, as well. FIG. 9D is a graph 951 that illustrates extreme variation among three different trials of index evolution with time in a subject, according to an embodiment. Horizontal axis 932 is the same as in FIG. 9B and FIG. 9C. The vertical axis 953 indicates the Neural Bias Index (NBI) value on a slightly different scale than in the preceding figures. Despite identical cue conditions (attend-left), three different single trials indicate strong moments of leftward attention (trace 956), rightward attention (trace 958), or no strong selective attention (trace 957).

Interestingly, as shown in FIG. 9B, the cue-specific modulation of the average NBI is also apparent for one subject at each individual 200 ms epoch during the trial. Due to inherent physiological and hardware noise associated with EEG-recordings, signal-to noise ratio was increased by employing a running average across multiple tone-pairs (1.25 s boxcar smoothing of about 6 successive tone-pairs) as shown in FIG. 9C, thus limiting this particular investigation to fluctuations in NBI on the scale of about 1 s or slower.

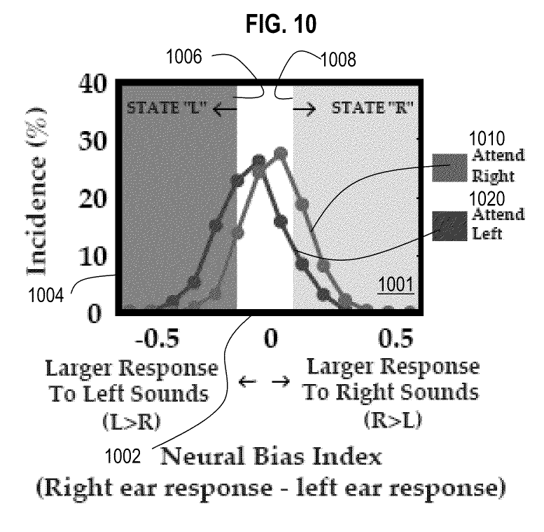

In contrast to the temporal stability in NBI throughout a trial on average, NBI time series within and across identically cued single trials were highly variable, as shown in FIG. 9D. While many trials did indeed demonstrate strong bias towards the cued ear (e.g., trace 956), other trials showed no bias (e.g., trace 957) or strong bias towards the non-cued ear (e.g., trace 958). In addition, these traces demonstrated rapid, endogenous fluctuations even within single trials, which were hypothesized to reflect rapid, implicitly driven shifts in attention within a trial, akin to neural correlates of explicitly cued shifts in attention observed in previous studies. The dispersion in instantaneous NBI within and between identically cued trials can also be observed in the broad distributions of neural bias index values during attend-left and attend-right cue conditions depicted later in FIG. 10.

Optimal brain states for detecting deviant tone were derived in step 317 by determining ranges of NBI values that appeared to discriminate lateral attention in the training set. Thresholds were determined for states of correctly or incorrectly directed attention towards or away from the cued ear, respectively, as follows: Using NBI values obtained during the training run(s), the estimated percentage of non-artifact trials containing correctly and incorrectly directed states were simulated for different values of upper and lower thresholds on the NBI. Threshold values were chosen such that correct/incorrect states (in the time interval from 2 s to 4 s after start of the series of tones) would each trigger deviant stimulus presentation on about 45% of trials. The selection of 45% incidences for each state was a trade-off between obtaining sufficient a number of triggered trials for statistical purposes versus including only mildly biased unilateral attention brain states.

FIG. 10 is a graph 1001 that illustrates index thresholds to define two brain states, according to an embodiment. The horizontal axis 1002 indicates NBI values. The vertical axis indicates a percent occurrence for each of multiple ranges of NBI values. The graph 1001 shows two histograms of occurrence of NBI values: attend R histogram 1010 for trials in which the subject was cued to attend to the right ear; and attend L histogram 1020 for trials in which the subject was cued to attend to the left ear, all in the training runs. A left attention brain state was defined as NBI values less than left threshold 1006. This left attention brain state occurs after 45% of tones when the subject is attending to the left ear, as indicated by histogram 1020. A right attention brain state was defined as NBI values greater than right threshold 1008. This right attention brain state occurs after 45% of tones when the subject is attending to the right ear, as indicated by histogram 1010.

The performance (e.g., behavioral response) influence of the extrema within these broad distributions of neural bias index values were assessed, as these instants in time could reflect extreme momentary biases in the subjects' attention towards one or the other ear. For purposes of real-time triggering, these (unimodal) distributions of neural bias index values were made discrete, identifying two “states” in which neural bias index values exceeded upper or lower thresholds (FIG. 2C,D). Thus, the neural bias index at each moment in time was classified as corresponding to a state of neural bias to the left ear or to the right ear (state “L”/state “R”, in FIG. 10, respectively), or to a state of low neural bias towards either ear. The threshold for an L or R state was selected such that, combined across both cue conditions, each state would occur in approximately 45% of trials (between 2-4 s post-train-onset). Interestingly, the choice of threshold levels involves an important compromise between targeting only the most pronounced neural bias states exceeding a high threshold (likely the most perceptually influential states), while maintaining a sufficiently low threshold to ensure an adequate number of trials containing states of interest.

In addition to these selection thresholds, triggering on extremely rare and unusually large ‘outlier’ NBI values was avoided by defining outer thresholds (not shown) for NBI values greater than +3 standard deviations from the overall mean NBI for the right attention brain state and less than −3 standard deviations from the overall mean NBI for the left attention brain state. Upon offline inspection, these rare occurrences of extreme NBI values often appeared to be caused by EEG channels contaminated by artifacts.

The actual percentages of trials in a stimulus triggered run containing each state was calculated following the run, and thresholds adjusted slightly to ensure equal incidence of left-ear and right-ear stimuli triggered by unilateral attention brain states (on average across cue conditions) during step 411.

Performance showed sensitivity to these brains states, e.g., as determined in step 411, described above. Several criteria were employed to exclude non-relevant or un-interpretable trials from the performance analyses. First, all non-triggered catch trials (˜10% of all trials), which could occur due to inattention, unbiased attention, or due to EEG artifacts, were excluded. There were two kinds of catch trials. In Triggered catch trials, a target brain state occurred, but instead of presenting a deviant tone, the standard tone was presented instead (thus, these trials provided a measure of false alarms). In non-triggered catch trials, the NBI never reaches criterion for triggering a target, due to unbiased or low laterality of pre-stimulus activity towards one ear, or because of EEG artifacts precluding assessment. These trials were, however, useful in encouraging subjects not to guess. In addition, trials were excluded in which performance (e.g., behavioral decisions) were strongly predicted by performance on recent trials (of same cue type), as the behavioral outcome in these trials would not reflect local, within-trial fluctuations in unilateral attention brain state. Specifically, it was observed that a marked increase in miss trials followed false-alarms; and so the next two trials following a false alarm were discarded from further analysis. In addition, hit/CR trials were omitted, which were both preceded and followed by a hit or CR. Similarly, miss/FA trials were omitted, which were both preceded and followed by a miss or FA, because these trials reflected epochs in which stimuli were far from the 50% detection threshold. These criteria resulted in exclusion, across subjects, of about 31% of all trials (min 23%, max 38% for individual subjects). The smaller final number of ‘usable’, artifact-free trials of comparable difficulty and brain state, further emphasizes the importance of brain state-triggered stimulus presentation for efficient study of ongoing activity.

Before addressing the effect of ongoing states on target detection, the robustness of the state-triggering algorithm was assessed.

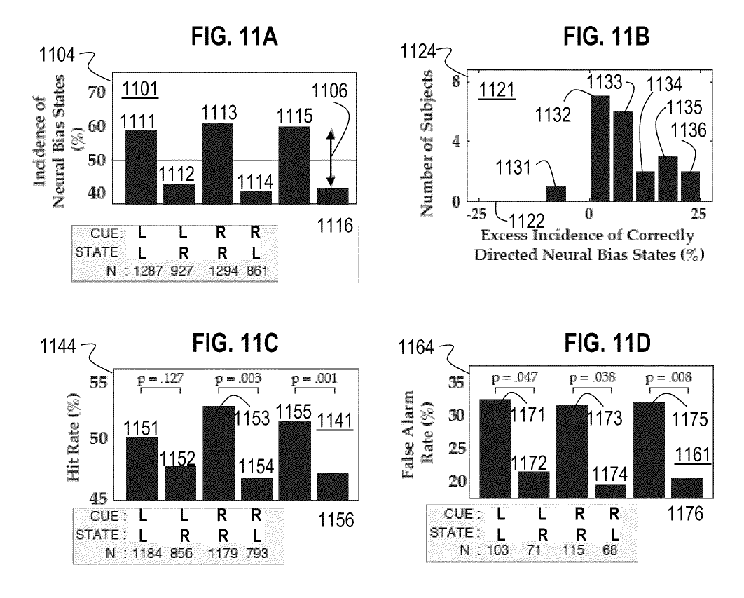

FIG. 11A is a bar graph 1101 that illustrates cue-induced incidence of brain states among multiple subjects, according to an embodiment. The vertical axis 1104 indicates the incidence of unilateral attention brain states, also called neural bias states herein, for all 21 subjects. The horizontal axis segregates different groupings of brain states. Bar 1111 indicates occurrence of left attention brain states in a trial in which the subject was cued to attend left. Bar 1112 indicates a lower occurrence of right attention brain states in a trial in which the subject was cued to attend left. Bar 1113 indicates occurrence of right attention brain states in a trial in which the subject was cued to attend right. Bar 1114 indicates a lower occurrence of left attention brain states in a trial in which the subject was cued to attend right. The side cued and the brain state side and the number of occurrences in each group are indicated below the bars 1111 through 1114.

As expected, a greater number of correctly vs. incorrectly directed neural bias states were observed, both for attend-left and attend-right cue conditions, as well as for data combined across conditions (% correctly directed attention brain states for attend-right trials: 60.05%, attend-left trials: 58.13%, combined: 59.08%). In other words, R states were more frequent than L states when subjects were cued to the right ear, and vice versa.

Brains states for attention to a side different from the cue side are less likely than brain states aligned with the cue. This point is emphasized by bar 1115 and bar 1116. Bar 1115 indicates the percentage of occurrences in which the brain state side is the same as the cue side, and bar 1116 indicates the percentage of occurrences in which the brain state side is opposite the cue side. The approximately 18% excess of aligned brain states is indicated by distance 1106. Thus, moments of correctly directed attention plotted as bar 1115 occurred more frequently than moments of incorrectly directed attention bar 1116.

FIG. 11B is a bar graph 1121 that illustrates distribution among subjects of excess incidence of brain states consistent with cue, according to an embodiment. The vertical axis 1124 indicates number of subjects. The horizontal axis 112 indicates the excess instances of aligned unilateral attention brain states in bins of 5 instances. All but one subject showed an excess of unilateral attention brain states aligned with the cued side. Thus, incidence of extreme states was modulated as expected by cue condition during triggered runs, despite freezing of parameters for calculating the neural bias index after initial training runs.

FIG. 11C is a bar graph 1141 that illustrates correct performance as a function of brain state and cuing, according to an embodiment. Note that deviant tones were presented only to the cued ear. The vertical axis 1144 indicates the hit rate, for all 21 subjects. The horizontal axis segregates different groupings of brain states. The hit rate is the number of correct detections (hits) divided by the total number of deviant tones in each grouping. Bar 1151 indicates hit rate for left attention brain states in a trial in which the subject was cued to attend left. Bar 1152 indicates hit rate for right attention brain states in a trial in which the subject was cued to attend left. Bar 1153 indicates hit rate for right attention brain states in a trial in which the subject was cued to attend right. Bar 1154 indicates hit rate for left attention brain states in a trial in which the subject was cued to attend right. The side cued and the brain state side and the number of occurrences in each group are indicated below the bars 1151 through 1154

Brains states for attention to a side different from the cue side are less likely score a hit. This point is emphasized by bar 1155 and bar 1156. Bar 1155 indicates the hit rate when the brain state side is the same as the cue side, and bar 1156 indicates the lower hit rate when the brain state side is opposite the cue side. As expected, the hit rate is better when the subject's attention is on the side where the deviant tone is presented. Despite identical cue conditions, detection of a deviant tone at the cued ear (‘hit rate’) was higher when the deviant tone was triggered by a correctly vs. incorrectly directed attention state.

By the same token, across cue conditions, the detection rate (hits/(hits+misses)) was significantly greater when the neural bias state was directed towards the cued ear (4.40% greater, P=0.001, non-parametric shuffle test, N=21 subjects, FIG. 11C). When considering individual cue conditions, a significant increase in hit rate was observed in the attend-right cue condition for R vs. L states (6.31% increase, P=0.003), and a similar but non-significant trend for the attend-left cue condition for L vs. R states (2.54% increase, P=0.127). Thus, instants of strong neural bias towards the correct vs. incorrect ear do influence subsequent detection performance on identically cued trials. These data further show that pre-target brain state-related effects on performance are spatially specific and not related to global effects, such as arousal. In other words, the presence of the same state (e.g., a state of strong pre-target neural bias towards the right ear) had opposite effects on target detection for attend-left and attend-right cue conditions.

Catch trials in which a strong pre-target neural bias state did not trigger deviant stimulus presentation were also analyzed. It was observed that significant differences in false alarm rates (false alarms/(false alarms+correct rejects)) depending on which state occurred during that trial. Specifically, subjects mistakenly reported hearing deviant target sounds more often on trials containing correctly vs. incorrectly directed states (attend right cue condition: 12.19% higher false alarm rate, P=0.038, attend left: 10.91%, P=0.047, combined: 11.51%, P=0.008, N=21 subjects, non-parametric shuffle test). These data suggest that ear-specific ‘hallucinations’ may be driven in part by instants of focused attention directed towards the cued ear.

FIG. 11C is a bar graph 1161 that illustrates a performance error as a function of brain state and cuing, according to an embodiment. The vertical axis 1164 indicates the false alarm rate, for all 21 subjects. The horizontal axis segregates different groupings of brain states. The false alarm rate is the number responses indicating a deviant tone when none was presented (false alarms) divided by the total number of non deviant tones in each grouping. Bar 1171 indicates false alarm rate for left attention brain states in a trial in which the subject was cued to attend left. Bar 1172 indicates false alarm rate for right attention brain states in a trial in which the subject was cued to attend left. Bar 1173 indicates false alarm rate for right attention brain states in a trial in which the subject was cued to attend right. Bar 1174 indicates false alarm rate for left attention brain states in a trial in which the subject was cued to attend right. The side cued and the brain state side and the number of occurrences in each group are indicated below the bars 1171 through 1174

Brain states for attention to a side different from the cue side are less likely result in a false alarm. This point is emphasized by bar 1175 and bar 1176. Bar 1175 indicates the false alarm rate when the brain state side is the same as the cue side, and bar 1176 indicates the lower false alarm rate when the brain state side is opposite the cue side. Surprisingly, the false alarm rate is worse (i.e., higher) when the subject's attention is on the side without a deviant tone. Interestingly, reported detection of targets on catch trials lacking deviants sounds (‘false alarm rate’) was also increased following strong attention towards the cued ear. Note that same unilateral attention brain state had opposite effects on behavior for attend-left and attend-right cue conditions. As demonstrated in this embodiment, target (deviant) stimuli were detected more often when triggered following moments of neural bias directed towards vs. away from the cued ear. Brain-state triggered stimulus delivery will enable efficient, statistically tractable studies of the influence of rare patterns of ongoing activity in single neurons and distributed neural circuits on subsequent behavioral and neural responses. Once the influence of these brain states are derived, they can be utilized to provide enhanced training or more intelligent sensory prostheses.

The state-specific increases in both behavioral detection and false-alarm rates ultimately carry opposite consequences for target discriminability. An estimate of discriminability was calculated for each cue/state combination, pooled across all trials and subjects. Surprisingly, the target stimuli were more readily discriminated in both cue conditions when the subjects' prior brain state was incorrectly directed away from the triggered ear (attend-right cue: d′ for incorrect vs. correct state: 0.79 vs. 0.56; attend-left: 0.74 vs. 0.47; combined: 0.76 vs. 0.51). This finding may be explained in part by the comparatively greater increase in false-alarm rates than in hit-rates following moments of correctly directed neural bias as shown in FIG. 11C and FIG. 11D.

Unless otherwise stated, statistical tests were unpaired t-tests. For comparison of response rates (FIG. 11C and FIG. 11D), a non-parametric shuffle test was employed. For example, a hit rate was considered significantly greater in condition A (N1 trials) than condition B (N2 trials) if the difference in actual hit rate exceeded the difference in shuffled hit rate at least 95% of the time. A distribution of 1000 shuffled hit rates was obtained by combining all trials in A and B, shuffling, reassigning N1 trials to A′ and N2 trials to B′, and re-computing hit rates in A′ and B′. According to the method of Green and Swets (1966), discriminability index d′ was defined. A corrected estimate of the discriminability of deviant tones from standard tones given a non-zero likelihood of false-alarms, was defined as d′=z(hit rate)−z(false alarm rate), where z( ) is the inverse of the cumulative normal distribution with mean=0 and standard deviation=1.

The above experiment represents a proof-of-principle demonstration that real time detected differences in ongoing brain activity are correlated with behavioral performance. A method was deliberately used where the ‘pre-target state’ was assessed by changes in the relative brain response magnitude following left vs. right ear tones. This ensured that the dynamic estimate of ear bias in processing was highly similar from subject to subject, stable throughout the course of each experiment, and interpretable as the result of selective processing of inputs from a specific ear. While the states examined here helped explain some of the trial-to-trial variability in behavioral performance, considerable behavioral variability remained (as shown, e.g., in FIG. 11). Some of this variability could potentially be explained by other overlapping pre-target brain states, such as non-ear-specific brain oscillations reflecting global or modality-specific vigilance or arousal. Therefore, we performed post-hoc analysis of the influence of pre-target oscillatory activity on perception.

Offline analysis of average EEG power across subjects revealed significant decreases in power in the one second prior to ‘hit’ trials vs. ‘miss’ trials, for both attend-left and attend-right conditions for the gamma band (60-100 Hz band). This oscillatory activity may reflect generalized states of concentration or arousal, and could be used in combination with unilateral attention brain states in the future.

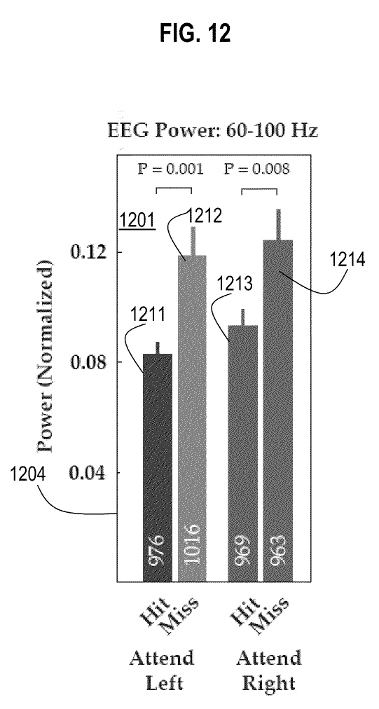

FIG. 12 is a bar graph 1201 that illustrates elevated high gamma activity increases miss rate regardless of cueing, according to an embodiment. The vertical axis 1204 indicates EEG normalized power in the 60 to 100 Hz band. Data were normalized by mean power across conditions for each subject prior to group averages. The horizontal axis segregates different groupings of performance by cue conditions. White numbers inside bars indicate total number of trials per condition, summed across twenty-one subjects. Thin error bars indicate standard error. Bar 1211 indicates the pre-stimulus power for brain state triggered deviant tones for which the subject scored a hit. Bar 1212 indicates the pre-stimulus power for brain state triggered deviant tones for which the subject scored a miss. Bar 1211 and bar 1212 are for left cued conditions. Bar 1213 and bar 1214 are similar to bar 1211 and bar 1212, respectively, but for right cued conditions.

We found that EEG power at fronto-temporal electrode sites F7 and F8 in the high gamma range (60-100 Hz, multi-taper spectral analysis) was significantly lower in the 1 s interval preceding ‘hit’ trials vs. ‘miss’ trials, for both attend-left and attend-right cue conditions (attend-left: P=0.001; attend-right: P=0.008, combination of trials across 21 subjects) as shown in FIG. 12. Similar but somewhat weaker effects were observed on the majority of electrodes (data not shown). Thus, in striking contrast to ear-specific bias states, which carried opposite behavioral consequences depending on which ear was cued (FIG. 11C), increases in gamma oscillatory activity led to increases in response rate irrespective of cue conditions (FIG. 12). Other frequency bands demonstrated less consistent effects across subjects that were not statistically significant (data not shown).

The illustrated embodiment demonstrated an estimated doubling in efficiency of recording trials involving coincidences of an ongoing state with the target stimulus, enabling an equal number of relevant trials to be collected in half the time, a major improvement given the limited duration of non-invasive recordings in humans. However, far greater gains in efficiency will be obtained by applying state-triggered stimulus presentation in studies using improved recording methods responsive to the shorter duration brain states depicted in FIG. 6. In this context, triggering stimuli on states of short duration and sparse occurrence (e.g. 200 ms states present 5% of the time) should lead to an order of magnitude increase in efficiency compared with traditional stimulus presentation schedules.

Another important consideration when studying the brain states correlated to performance (e.g., lateralized detection) is the choice of neural indicator function and task. The neural indicator function used (bias index) was easy to calculate and was modulated strongly by cue condition in a manner consistent across subjects, simplifying real time extraction of states with relatively brief training time. To increase cue-dependent modulation of lateralized neural bias, a difficult detection task was engaged using near-threshold target stimuli and high-rates of sound presentation (5 Hz). Note that correct detection rates (FIG. 11C) were roughly twofold greater than false alarm rates (FIG. 11D), suggesting that subjects performed well above chance on this deliberately difficult task

The illustrated embodiment is unique in that ongoing fluctuations in neural bias, likely reflecting, in part, fluctuations in selective listening were used to trigger presentation of sensory stimuli in real time.

The process presented here differs from previous ‘neuro-feedback’ studies that aim to treat disorders of attention or cognition by asking subjects to regulate their brain activity in various frequency bands towards ‘normal’ levels. Modulation of brain activity in these neuro-feedback studies does not occur in the context of sustained performance of a well-defined task, rendering it more difficult to infer the source(s) of induced oscillatory activity and less useful for specific training regimens.

It is expected in further embodiments, to trigger target stimuli conditionally on brain signals from different spatial locations. Indeed, the brain-state triggered stimulus delivery method presented here is quite general, and could be used to efficiently probe and exploit the interaction between evoked neural and/or behavioral responses with complex patterns of sparse ongoing brain activity recorded from ensembles of individual neurons using multi-electrodes or two-photon calcium imaging in vivo and in vitro, among other new and evolving brain signal measuring technologies.

### Example Hardware

The processes described herein for triggering a stimulus based on brain state may be implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such example hardware for performing the described functions is detailed below

FIG. 13 illustrates a computer system 1300 upon which an embodiment of the invention may be implemented. Computer system 1300 includes a communication mechanism such as a bus 1310 for passing information between other internal and external components of the computer system 1300. Information (also called data) is represented as a physical expression of a measurable phenomenon, typically electric voltages, but including, in other embodiments, such phenomena as magnetic, electromagnetic, pressure, chemical, biological, molecular, atomic, sub-atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range.

A bus 1310 includes one or more parallel conductors of information so that information is transferred quickly among devices coupled to the bus 1310. One or more processors 1302 for processing information are coupled with the bus 1310.

A processor 1302 performs a set of operations on information. The set of operations include bringing information in from the bus 1310 and placing information on the bus 1310. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 1302, such as a sequence of operation codes, constitute processor instructions, also called computer system instructions or, simply, computer instructions. Processors may be implemented as mechanical, electrical, magnetic, optical, chemical or quantum components, among others, alone or in combination.

Computer system 1300 also includes a memory 1304 coupled to bus 1310. The memory 1304, such as a random access memory (RAM) or other dynamic storage device, stores information including processor instructions. Dynamic memory allows information stored therein to be changed by the computer system 1300. RAM allows a unit of information stored at a location called a memory address to be stored and retrieved independently of information at neighboring addresses. The memory 1304 is also used by the processor 1302 to store temporary values during execution of processor instructions. The computer system 1300 also includes a read only memory (ROM) 1306 or other static storage device coupled to the bus 1310 for storing static information, including instructions, that is not changed by the computer system 1300. Some memory is composed of volatile storage that loses the information stored thereon when power is lost. Also coupled to bus 1310 is a non-volatile (persistent) storage device 1308, such as a magnetic disk, optical disk or flash card, for storing information, including instructions, that persists even when the computer system 1300 is turned off or otherwise loses power.

Information, including instructions, is provided to the bus 1310 for use by the processor from an external input device 1312, such as a keyboard containing alphanumeric keys operated by a human user, or a sensor. A sensor detects conditions in its vicinity and transforms those detections into physical expression compatible with the measurable phenomenon used to represent information in computer system 1300. Other external devices coupled to bus 1310, used primarily for interacting with humans, include a display device 1314, such as a cathode ray tube (CRT) or a liquid crystal display (LCD), or plasma screen or printer for presenting text or images, and a pointing device 1316, such as a mouse or a trackball or cursor direction keys, or motion sensor, for controlling a position of a small cursor image presented on the display 1314 and issuing commands associated with graphical elements presented on the display 1314. In some embodiments, for example, in embodiments in which the computer system 1300 performs all functions automatically without human input, one or more of external input device 1312, display device 1314 and pointing device 1316 is omitted.

In the illustrated embodiment, special purpose hardware, such as an application specific integrated circuit (ASIC) 1320, is coupled to bus 13 10. The special purpose hardware is configured to perform operations not performed by processor 1302 quickly enough for special purposes. Examples of application specific ICs include graphics accelerator cards for generating images for display 1314, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1300 also includes one or more instances of a communications interface 1370 coupled to bus 1310. Communication interface 1370 provides a one-way or two-way communication coupling to a variety of external devices that operate with their own processors, such as printers, scanners and external disks. In general the coupling is with a network link 1378 that is connected to a local network 1380 to which a variety of external devices with their own processors are connected. For example, communication interface 1370 may be a parallel port or a serial port or a universal serial bus (USB) port on a personal computer. In some embodiments, communications interface 1370 is an integrated services digital network (ISDN) card or a digital subscriber line (DSL) card or a telephone modem that provides an information communication connection to a corresponding type of telephone line. In some embodiments, a communication interface 1370 is a cable modem that converts signals on bus 1310 into signals for a communication connection over a coaxial cable or into optical signals for a communication connection over a fiber optic cable. As another example, communications interface 1370 may be a local area network (LAN) card to provide a data communication connection to a compatible LAN, such as Ethernet. Wireless links may also be implemented. For wireless links, the communications interface 1370 sends or receives or both sends and receives electrical, acoustic or electromagnetic signals, including infrared and optical signals, that carry information streams, such as digital data. For example, in wireless handheld devices, such as mobile telephones like cell phones, the communications interface 1370 includes a radio band electromagnetic transmitter and receiver called a radio transceiver.

The term computer-readable medium is used herein to refer to any medium that participates in providing information to processor 1302, including instructions for execution. Such a medium may take many forms, including, but not limited to, non-volatile media, volatile media and transmission media. Non-volatile media include, for example, optical or magnetic disks, such as storage device 1308. Volatile media include, for example, dynamic memory 1304. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a transmission medium such as a cable or carrier wave, or any other medium from which a computer can read. Information read by a computer from computer-readable media are variations in physical expression of a measurable phenomenon on the computer readable medium. Computer-readable storage medium is a subset of computer-readable medium which excludes transmission media that carry transient man-made signals.

Logic encoded in one or more tangible media includes one or both of processor instructions on a computer-readable storage media and special purpose hardware, such as ASIC 1320.

Network link 1378 typically provides information communication using transmission media through one or more networks to other devices that use or process the information. For example, network link 1378 may provide a connection through local network 1380 to a host computer 1382 or to equipment 1384 operated by an Internet Service Provider (ISP). ISP equipment 1384 in turn provides data communication services through the public, world-wide packet-switching communication network of networks now commonly referred to as the Internet 1390. A computer called a server host 1392 connected to the Internet hosts a process that provides a service in response to information received over the Internet. For example, server host 1392 hosts a process that provides information representing video data for presentation at display 1314.

At least some embodiments of the invention are related to the use of computer system 1300 for implementing some or all of the techniques described herein. According to one embodiment of the invention, those techniques are performed by computer system 1300 in response to processor 1302 executing one or more sequences of one or more processor instructions contained in memory 1304. Such instructions, also called computer instructions, software and program code, may be read into memory 1304 from another computer-readable medium such as storage device 1308 or network link 1378. Execution of the sequences of instructions contained in memory 1304 causes processor 1302 to perform one or more of the method steps described herein. In alternative embodiments, hardware, such as ASIC 1320, may be used in place of or in combination with software to implement the invention. Thus, embodiments of the invention are not limited to any specific combination of hardware and software, unless otherwise explicitly stated herein.

The signals transmitted over network link 1378 and other networks through communications interface 1370, carry information to and from computer system 1300. Computer system 1300 can send and receive information, including program code, through the networks 1380, 1390 among others, through network link 1378 and communications interface 1370. In an example using the Internet 1390, a server host 1392 transmits program code for a particular application, requested by a message sent from computer 1300, through Internet 1390, ISP equipment 1384, local network 1380 and communications interface 1370. The received code may be executed by processor 1302 as it is received, or may be stored in memory 1304 or in storage device 1308 or other non-volatile storage for later execution, or both. In this manner, computer system 1300 may obtain application program code in the form of signals on a carrier wave.

Various forms of computer readable media may be involved in carrying one or more sequence of instructions or data or both to processor 1302 for execution. For example, instructions and data may initially be carried on a magnetic disk of a remote computer such as host 1382. The remote computer loads the instructions and data into its dynamic memory and sends the instructions and data over a telephone line using a modem. A modem local to the computer system 1300 receives the instructions and data on a telephone line and uses an infra-red transmitter to convert the instructions and data to a signal on an infra-red carrier wave serving as the network link 1378. An infrared detector serving as communications interface 1370 receives the instructions and data carried in the infrared signal and places information representing the instructions and data onto bus 1310. Bus 1310 carries the information to memory 1304 from which processor 1302 retrieves and executes the instructions using some of the data sent with the instructions. The instructions and data received in memory 1304 may optionally be stored on storage device 1308, either before or after execution by the processor 1302.

FIG. 14 illustrates a chip set 1400 upon which an embodiment of the invention may be implemented. Chip set 1400 is programmed to carry out the inventive functions described herein and includes, for instance, the processor and memory components described with respect to FIG. 14 incorporated in one or more physical packages. By way of example, a physical package includes an arrangement of one or more materials, components, and/or wires on a structural assembly (e.g., a baseboard) to provide one or more characteristics such as physical strength, conservation of size, and/or limitation of electrical interaction.

In one embodiment, the chip set 1400 includes a communication mechanism such as a bus 1401 for passing information among the components of the chip set 1400. A processor 1403 has connectivity to the bus 1401 to execute instructions and process information stored in, for example, a memory 1405. The processor 1403 may include one or more processing cores with each core configured to perform independently. A multi-core processor enables multiprocessing within a single physical package. Examples of a multi-core processor include two, four, eight, or greater numbers of processing cores. Alternatively or in addition, the processor 1403 may include one or more microprocessors configured in tandem via the bus 1401 to enable independent execution of instructions, pipelining, and multithreading. The processor 1403 may also be accompanied with one or more specialized components to perform certain processing functions and tasks such as one or more digital signal processors (DSP) 1407, or one or more application-specific integrated circuits (ASIC) 1409. A DSP 1407 typically is configured to process real-word signals (e.g., sound) in real time independently of the processor 1403. Similarly, an ASIC 1409 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1403 and accompanying components have connectivity to the memory 1405 via the bus 1401. The memory 1405 includes both dynamic memory (e.g., RAM, magnetic disk, writable optical disk, etc.) and static memory (e.g., ROM, CD-ROM, etc.) for storing executable instructions that when executed perform the inventive steps described herein. The memory 1405 also stores the data associated with or generated by the execution of the inventive steps.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1 A method comprising:
receiving data that indicates a brain state and a set of one or more stimuli associated with the brain state;
detecting onset in a subject of an instance of the brain state; and
in response to detecting onset of the instance, initiating application to the subject of a stimulus of the set before the instance ends.

2. A method of claim 1, wherein detecting the onset in the subject of the instance of the brain state further comprises determining that a value of a function of one or more electrical signals detected at corresponding electrodes placed near the subject falls within a predetermined range of values.

3. A method of claim 1, wherein the set of one or more stimuli includes a gain for a device to assist perception of external phenomenon.

4. A method of claim 1, wherein:
the brain state is associated with a superior capacity to perceive a particular sensory input; and
the set of one or more stimuli includes the particular sensory input.

5. A method of claim 1, wherein:
the brain state is associated with a superior capacity to perform a particular function; and
the set of one or more stimuli includes an alert to the subject to attempt to perform the particular function.

6. A method of claim 5, wherein:
the particular function is memorization of a fact; and
the alert includes a presentation of the fact.

7. A method of claim 5, wherein the particular function is a movement.

8. A method of claim 1, wherein:
the brain state is associated with a superior capacity to perform a particular function; and
the set of one or more stimuli includes a gain for a device to assist the subject to perform the particular function.

9. A method of claim 8, wherein:
the particular function is a movement; and
the device causes the subject to execute the movement.

10. A method of claim 1, wherein the brain state is associated with a superior capacity to respond to a stimulus of the set based on measurements of performance of the subject's response to the stimulus and simultaneous measurements of one or more electrical signals detected at corresponding electrodes placed near the subject.

11. A method of claim 1, wherein the brain state is associated with a superior capacity to respond to a stimulus of the set based on measurements of performance of a different subject's response to the stimulus and simultaneous measurements of one or more electrical signals detected at corresponding electrodes placed near the different subject.

12. A method of claim 1, wherein the instance of the brain state ends within about ten seconds of the onset of the instance of the brain state.

13. A method of claim 1, wherein the instance of the brain state ends within about one second of the onset of the instance of the brain state.

14. A method of claim 2, wherein initiating application to the subject of the stimulus before the instance ends further comprises initiating application of the stimulus at a particular phase of an oscillation in the electrical signal that persists during the brain state.

15. A method of claim 1, wherein:
the brain state is more likely to occur in response to a different stimulus; and
the method further comprises initiating application to the subject of the different stimulus.

16. A computer-readable storage medium carrying one or more sequences of one or more instructions which, when executed by one or more processors, cause the one or more processors to perform:
receiving data that indicates a brain state and a set of one or more stimuli associated with the brain state;
detecting onset in a subject of an instance of the brain state; and
in response to detecting onset of the instance, initiating application of a stimulus of the set before the instance ends.

17. An apparatus configured to:
receive data that indicates a brain state and a set of one or more stimuli associated with the brain state;
detect onset in a subject of an instance of the brain state; and
in response to detecting onset of the instance, initiate application of a stimulus of the set before the instance ends.

18. An apparatus comprising:
means for receiving data that indicates a brain state and a set of one or more stimuli associated with the brain state;
means for detecting onset in a subject of an instance of the brain state; and
means for initiating application of a stimulus of the set before the instance ends, in response to detecting onset of the instance.

19. A method comprising:
receiving signal data that indicates one or more electrical signals detected at corresponding electrodes placed near a first subject;
receiving performance data indicating response of the first subject to a stimulus during a time interval included in the signal data;
determining desired performance within the performance data;
determining a brain state based on a range of values for a function of the signal data, wherein the range of values is associated with the desired performance; and
causing the stimulus to be presented to a second subject when the brain state is detected in the second subject.

20. A method of claim 19, wherein the second subject is the same as the first subject.
